(19) United States
(12) Patent Application Publication
(10) Pub. No.: US 2008/0200679 A1
(43) Pub. Date: Aug. 21, 2008

(54) Alpha-HYDROXY, alpha-SUBSTITUTED METHYLENEBISPHOSPHONATES AND PHOSPHONOACETATES

(75) Inventors: Charles E. McKenna, Pacific Palisades, CA (US); Boris A. Kashemirov, Los Angeles, CA (US); Gregorio V. Sanchez, Pasadena, CA (US)

Correspondence Address: HOGAN & HARTSON, Suite 1400, 1999 Avenue of the Stars, Los Angeles, CA, 90067, US

(73) Assignee: UNIVERSITY OF SOUTHERN CALIFORNIA, Los Angeles, CA (US)

(21) Appl. No.: 12/018,121

(22) Filed: Jan. 22, 2008

Related U.S. Application Data
(60) Provisional application No. 60/886,036, filed on Jan. 22, 2007.

(51) Int. Cl. C07F 7/02; C07F 9/40; C07D 213/28; C07F 9/655; C07F 9/38

(52) U.S. Cl. 546/14; 558/89; 568/14; 546/22; 549/216

(57) ABSTRACT

The present invention relates to a method for preparing α-hydroxy, α-substituted methylenebisphosphonates and phosphonoacetates via addition of Grignard or organoindium reagents to tetraalkyl carbonylbisphosphonates and trialkyl carbonylphosphonoacetates. Also disclosed are compounds so synthesized.

### RELATED APPLICATION

This application claims priority to U.S. Provisional Patent Application Ser. No. 60/886,036, filed on Jan. 22, 2007, the content of which is incorporated herein by reference in its entirety.

### FIELD OF THE INVENTION

The present invention relates in general to α-hydroxy, α-substituted methylenebisphosphonates and phosphonoacetates. More specifically, the invention provides methods for the synthesis of α-hydroxy, α-substituted methylenebisphosphonate and phosphonoacetate esters and acid salts via addition of Grignard or organoindium reagents to tetraalkyl carbonylbisphosphonates and trialkyl carbonylphosphonoacetates.

### BACKGROUND OF THE INVENTION

Biological phosphonate analogs have been of interest as pharmaceutical targets since the 1970's. Bisphosphonate (BP) and phosphonoacetate (PA) derivatives have been used in the treatment of bone diseases^{1, 2}, cancer^{3, 4}, and viral therapies⁵⁻⁷. Inherent in both analogs is the bridging methylene carbon (α-carbon), which can be functionalized to thereby fine-tune biological activity. It has been well established that incorporating a methylene-linked nitrogen-containing heterocycle at the α-carbon of an α-hydroxy methylenebisphosphonate (α-HRMBP) increases binding affinity to the farnesyl diphosphate synthase (FPPS) active site⁸⁻¹⁰. In addition, studies have also revealed that certain heterocyclic derivatives of α-HRMBP and α-hydroxy phosphonoacetates (α-HRMPA) inhibit Rab geranylgeranyl transferase, disrupting the prenylation of Rab proteins in osteoclasts¹¹.

Conventional synthetic methodologies for both analogs consist of the reaction of a carboxylic acid (RCO₂H) with various phosphorylating reagents under harsh conditions¹²⁻¹⁷. With respect to the BP, milder routes through ester intermediates have also been explored¹⁸⁻²⁰. Unlike the more stable α-HRMBP salts²¹, ester derivatives have been observed to rearrange to the corresponding phosphonophosphate (P—O—C—P) (Scheme 1.1) or fragment into a phosphite and ketone, both of which are acid, base, and thermally catalyzed²¹⁻²⁶. Furthermore, basic nucleophiles, when reacted with α-keto acylphosphonates, were also shown to promote dephosphorylation^{24, 27}. This presents a major obstacle in the utilization of BP esters as synthons to novel and currently administered BP.

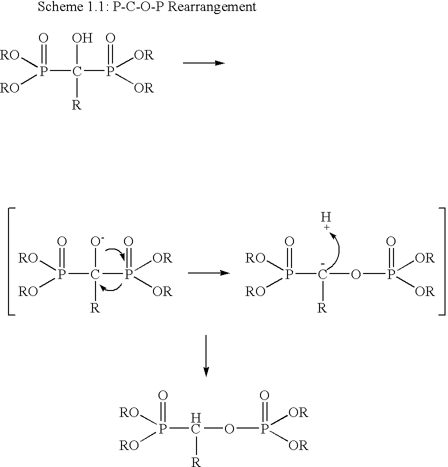

The synthesis of alkyl esters of carbonylphosphonoacetate and carbonylbisphosphonate (Scheme 1.2)²⁸⁻³¹ has been reported, which, in principle, makes possible a mild approach to α-HRMBP and α-HRMPA esters via addition of nucleophilic species to the reactive α-ketone group (Scheme 1.3). Nucleophilic addition studies with water and methanol confirmed that triethyl α-keto phosphonoacetate (COTEPA) and α-keto tetraisopropyl methylenebisphosphonate (iPr₄COBP) are reactive^{28, 32}. Decomposition to phosphite and ketone in both COTEPA and iPr₄COBP produces pentaalkyl phosphonoacetate (dimer) and hexaalkyl bisphosphonophosphate (trimer), respectively (Scheme 1.4)³².

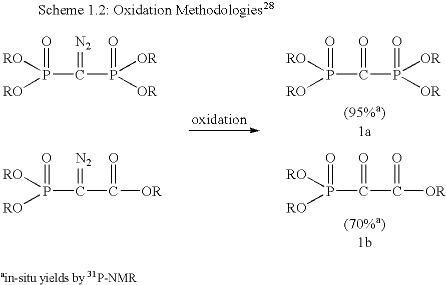

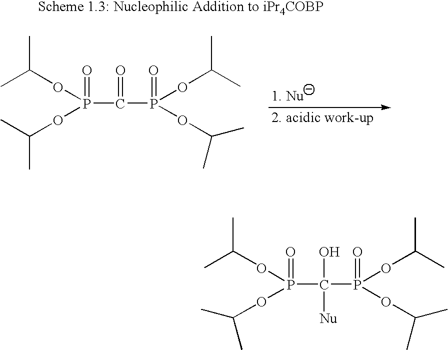

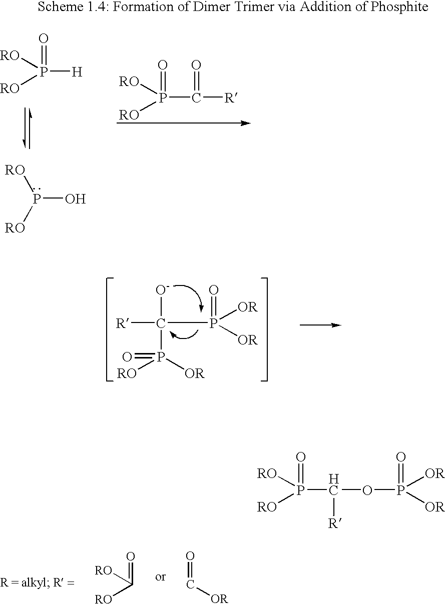

### SUMMARY OF THE INVENTION

This invention is based, at least in part, on the unexpected discovery that simple alkyl and aryl Grignard reagents, and also indium mediated Barbier allyl derivatives, add to iPr₄COBP and COTEPA with minimal rearrangement or decomposition. In cases where iPr₄COBP and COTEPA are contacted with heteroatom-containing Grignard reagents generated at low temperatures, rearrangement can be reduced by adding chlorotrimethylsilane (CTMS) to protect the α-hydroxy group. This method has provided a route to the first examples of heteroatom-containing silyl-protected α-HRMBP and α-HRMPA esters via nucleophilic addition. Facile silyldealkylation using bromotrimethylsilane (BTMS) in acetonitrile provides the α-HRMBP and α-HRMPA acids in good to fair yields.

Accordingly, one method of the invention for preparing a compound comprises contacting iPr₄COBP with a Grignard reagent to form an alkyl- or aryl-substituted α-HRMBP ester, which may then be converted to the corresponding acid or salt using a method well known in the art or described in the present application. The Grignard reagent is generated in Et₂O:THF, the ratio of which is in the range of 5:1 to 1:5, and preferably is 1:1. In some embodiments, the Grignard reagent is RMgX, R being an alkyl or aryl group and X being a halo group. Examples of R include, but are not limited to, a methyl, phenyl, or benzyl group.

Another method of the invention for preparing a compound comprises contacting iPr₄COBP or COTEPA with a Grignard reagent followed by an anion-interceptor to form a substituted α-HRMBP or α-HRMPA ester, which may then be converted to the corresponding acid or salt using a method well known in the art or described in the present application. The anion-interceptor may be a silylating agent, for example, CTMS.

In some embodiments, the Grignard reagent is a heterocyclic Grignard reagent. For example, the Grignard reagent may contain nitrogen in the heterocyclic group, such as a Grignard reagent derived from 2-chloropyridine. The substituted α-HRMBP or α-HRMPA ester may be converted into its corresponding acid in the presence of acetonitrile. In some embodiments, the Grignard reagent is an alkyl or aryl Grignard reagent.

The invention further provides a method of preparing a compound comprising contacting iPr₄COBP or COTEPA with an unsaturated indium halide reagent (e.g., allyl InBr₂) to form an unsaturated substituted α-HRMBP or α-HRMPA ester, which may then be converted to the corresponding acid or salt using a method well known in the art or described in the present application. For example, the unsaturated substituted α-HRMBP ester may be converted into its corresponding acid in the presence of acetonitrile.

Examples of an allylic group include, but are not limited to:

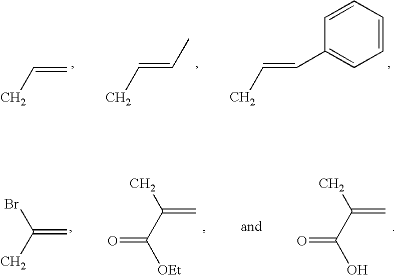

In one embodiment,

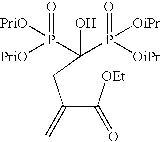

may cyclize to form

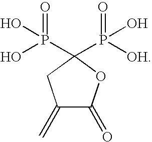

In some embodiments, iPr₄COBP or COTEPA is contacted with the unsaturated indium halide reagent in the presence of acetic acid or CTMS. In some embodiments, iPr₄COBP or COTEPA is contacted with the unsaturated indium halide reagent in the presence of a Lewis acid such as BF₃. etherate.

A compound prepared according to a method described above is within the invention. For example, the invention provides a compound of formula

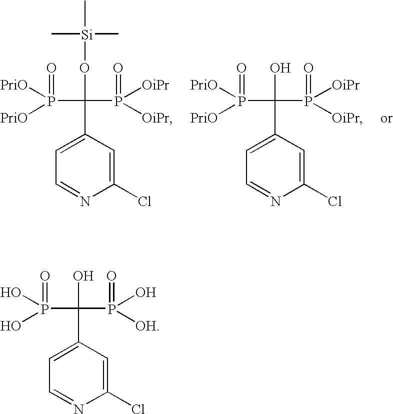

The invention also provides an unsaturated substituted α-HRMBP or α-HRMPA ester, or the corresponding acid or salt, wherein the unsaturated substitution group is

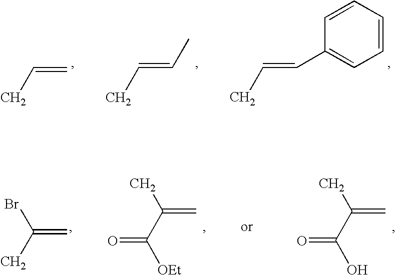

as well as a compound of formula

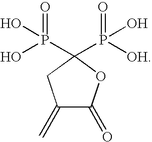

The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIGS. 1A-C. Bisphosphonates

FIGS. 2A-B. Tetraalkyl Carbonylbisphosphonate

FIGS. 3A-B. Decomposition Pathway

FIG. 4. Grignard Addition to Tetraalkyl Carbonylbisphosphonate

FIG. 5. Overview

FIG. 6. Alkyl and Aryl Substituted α-HRMBP

FIG. 7. Nitrogen-containing α-HRMBP-Rearrangement

FIGS. 8A-C. Nitrogen-containing α-HRMBP

FIGS. 9A-D. Indium-promoted Synthesis of Unsaturated α-HRMBP.

FIG. 10. Cyclization of Acrylate Derivative upon Dealkylation

FIGS. 11A-B. Alternative Organometallic Reagents

FIG. 12—Conclusions

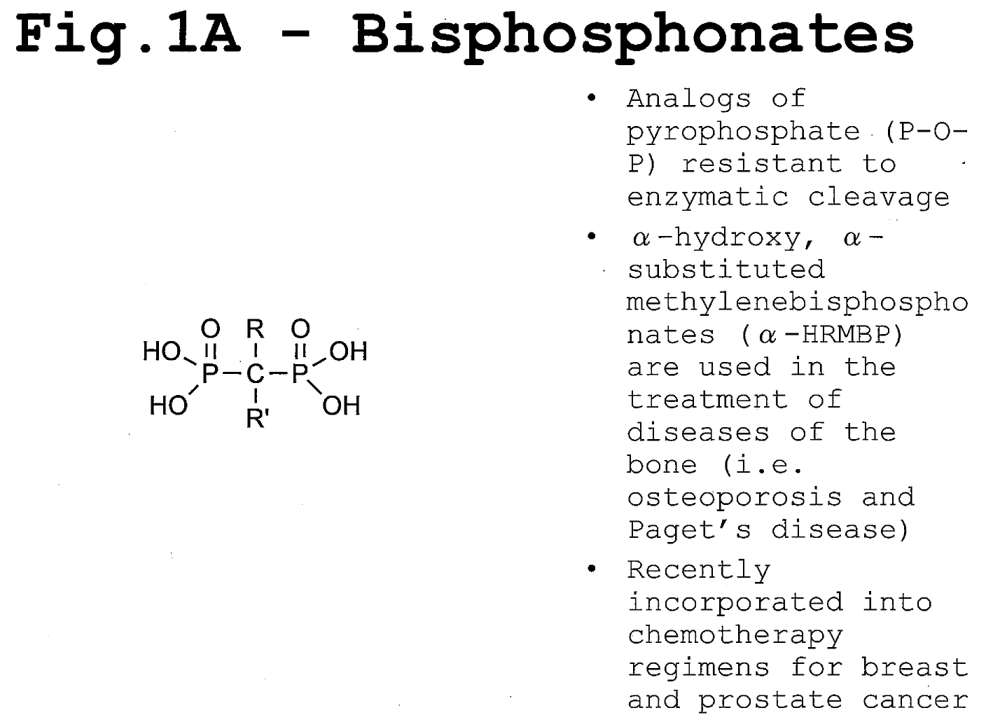

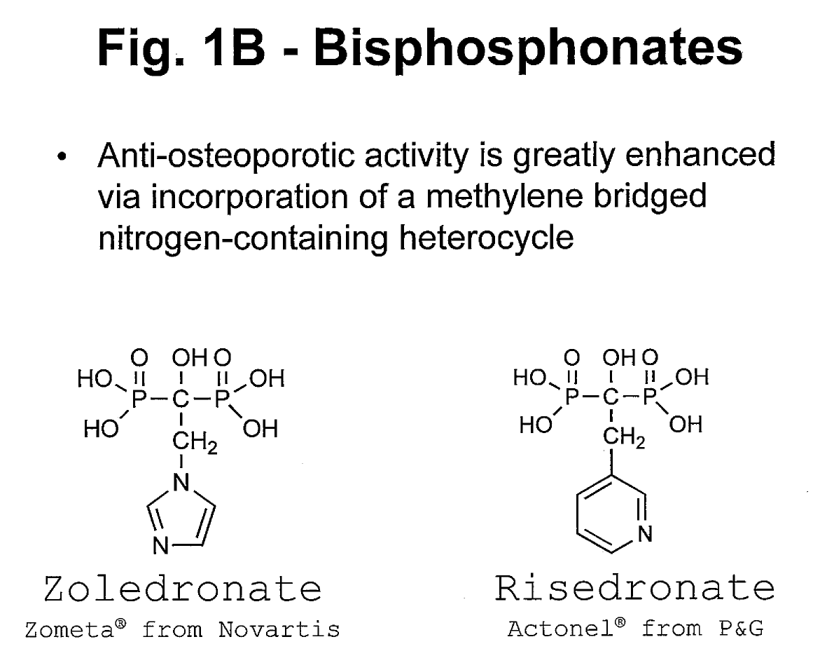

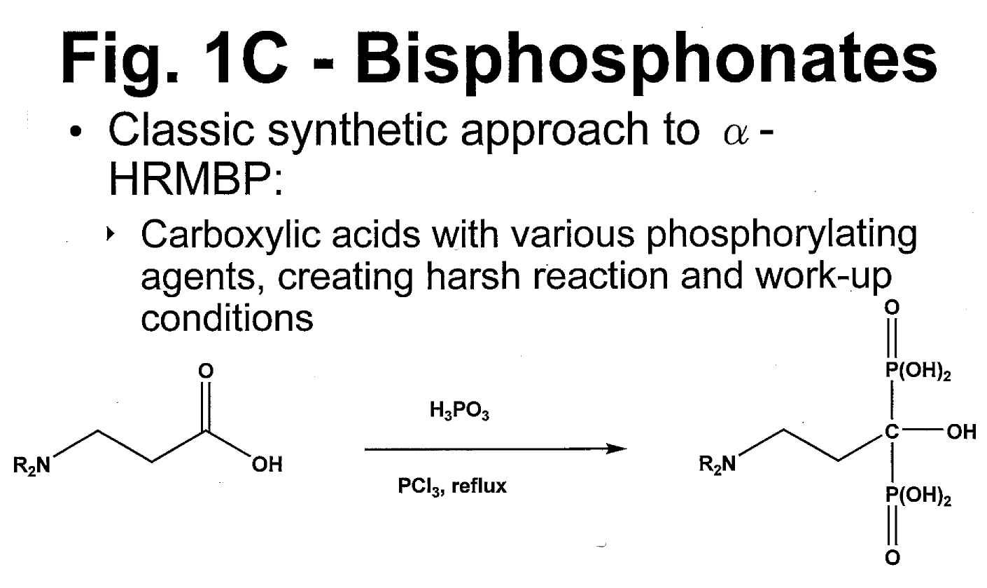

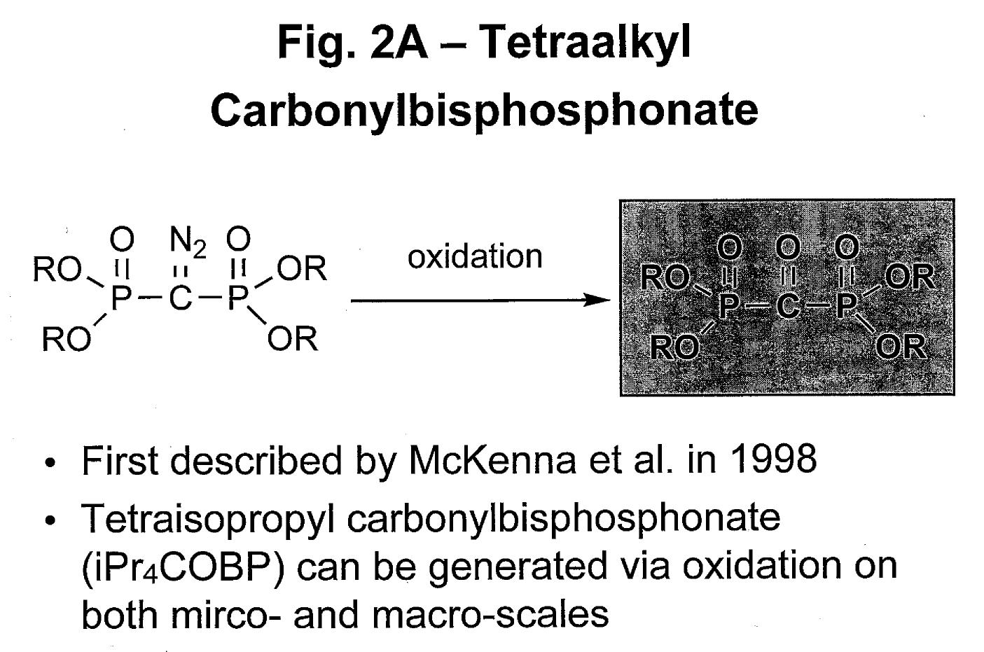

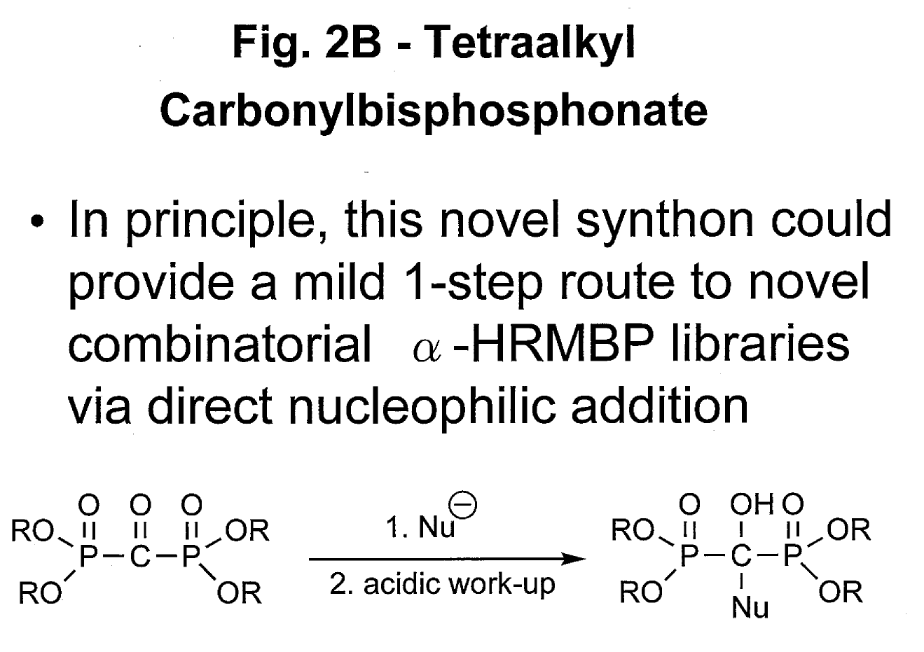

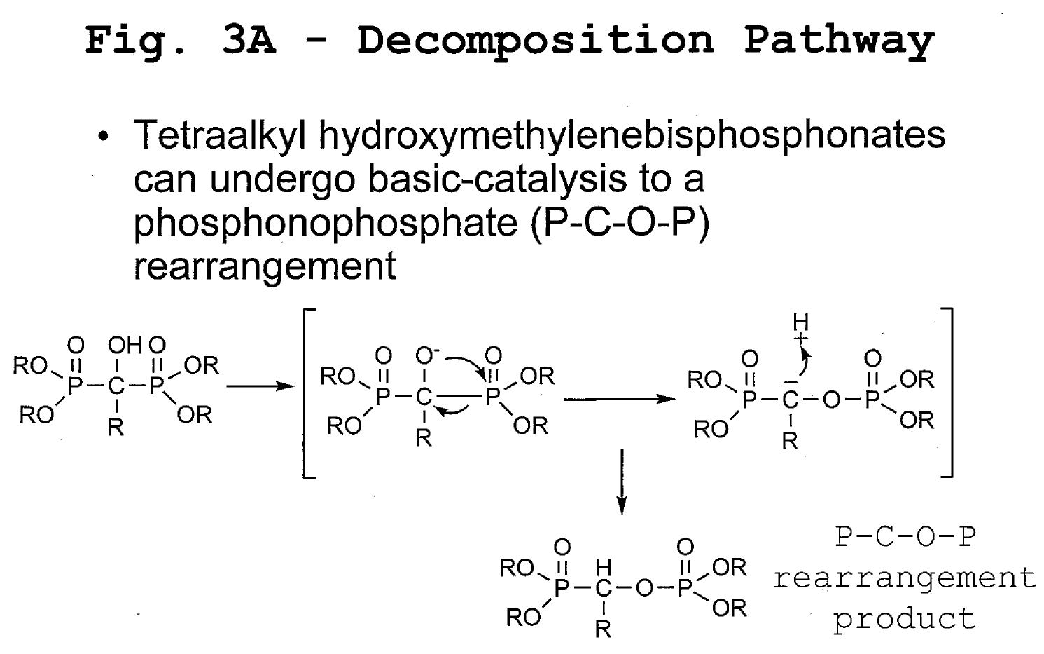

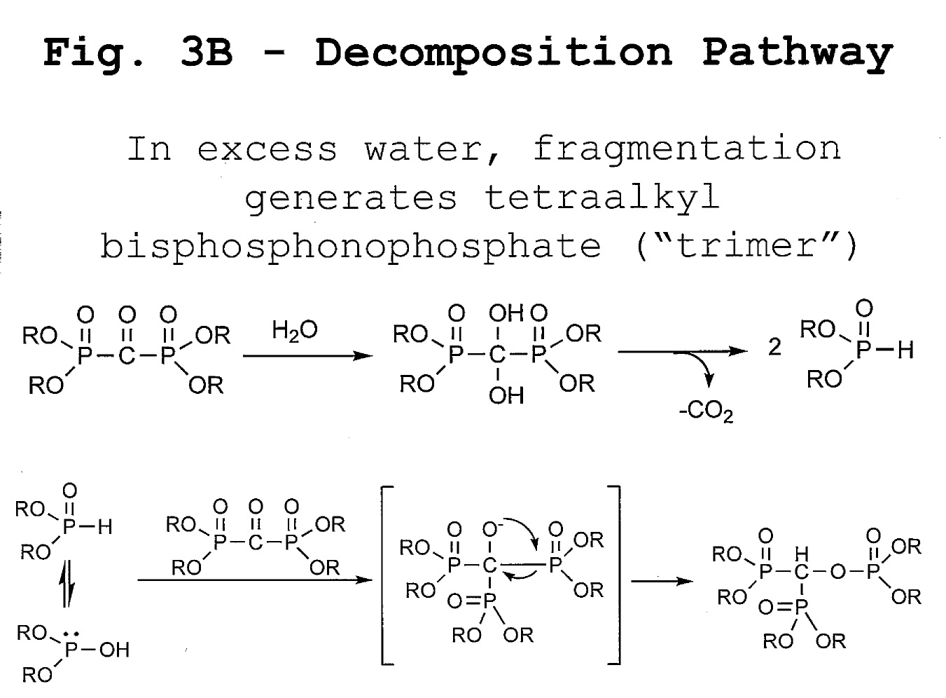

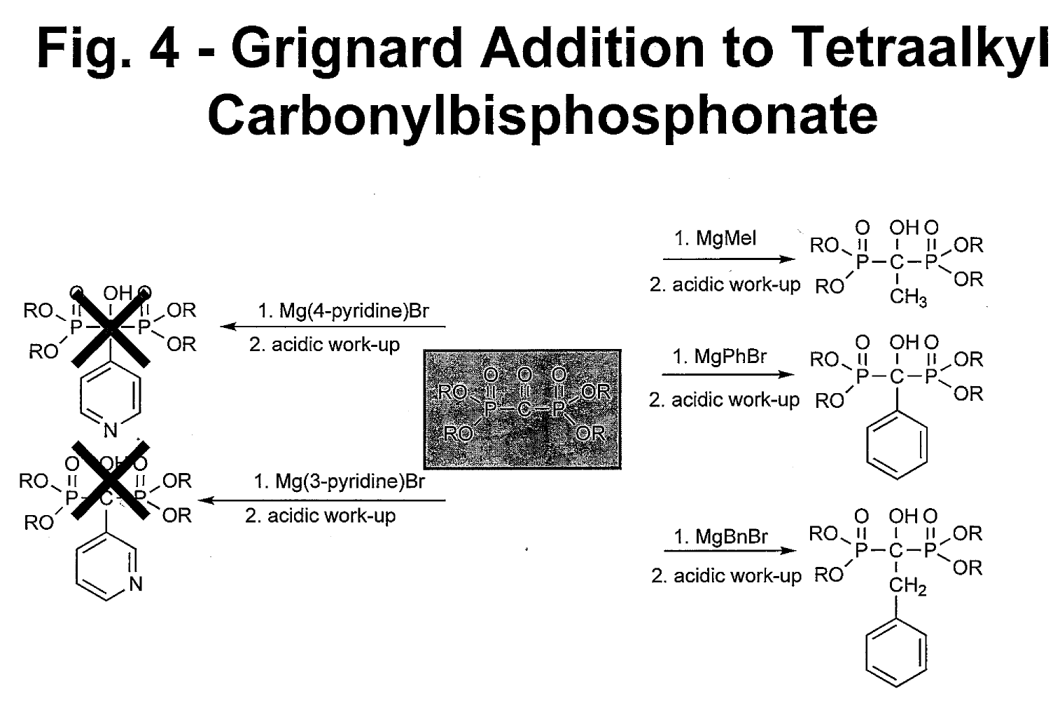

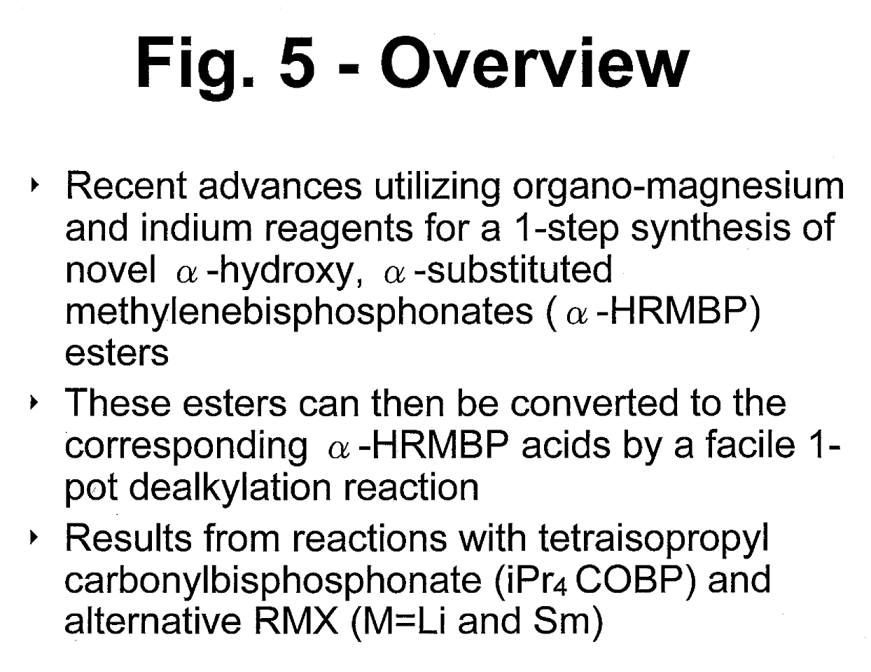

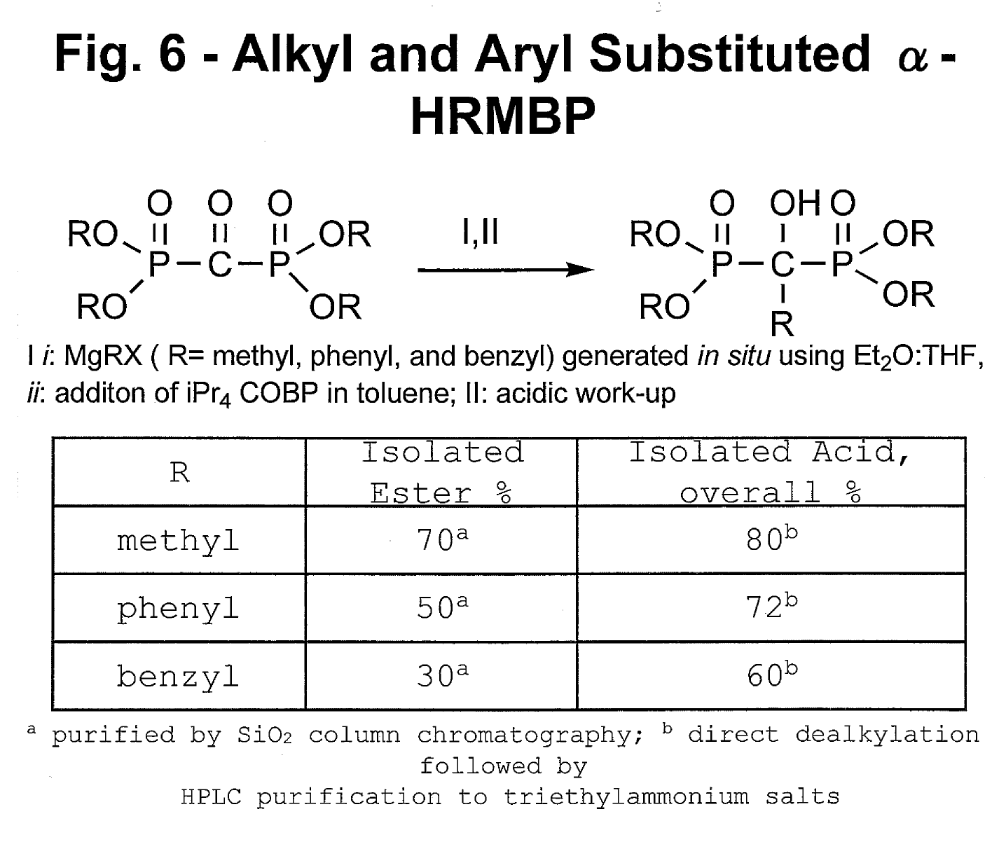

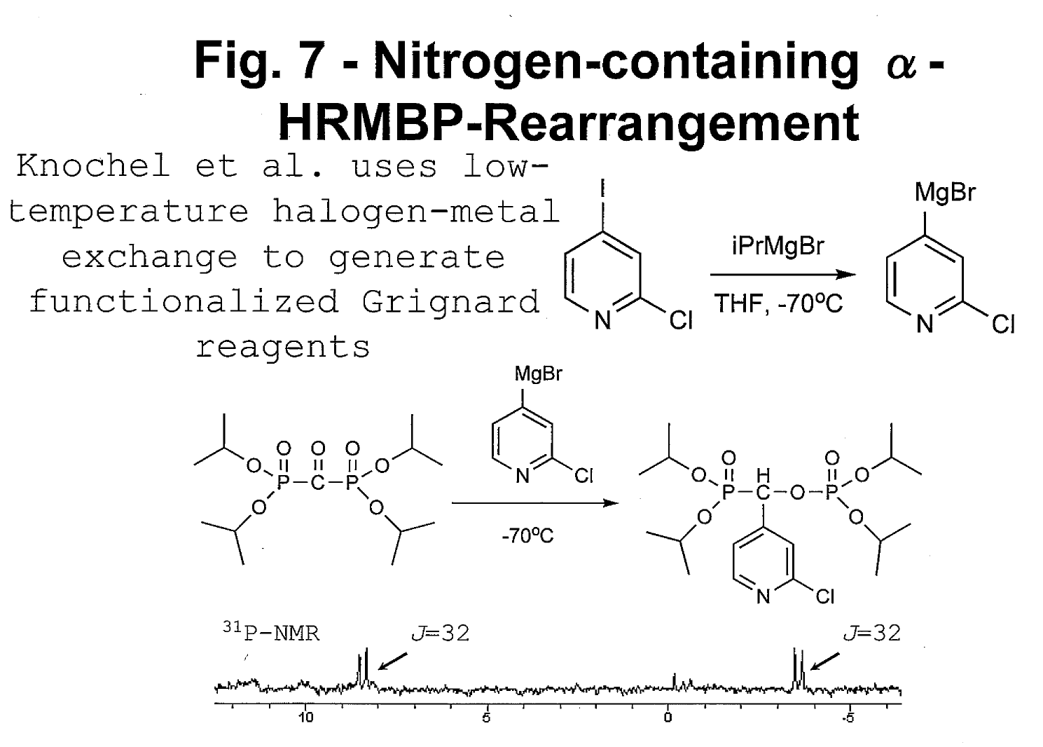

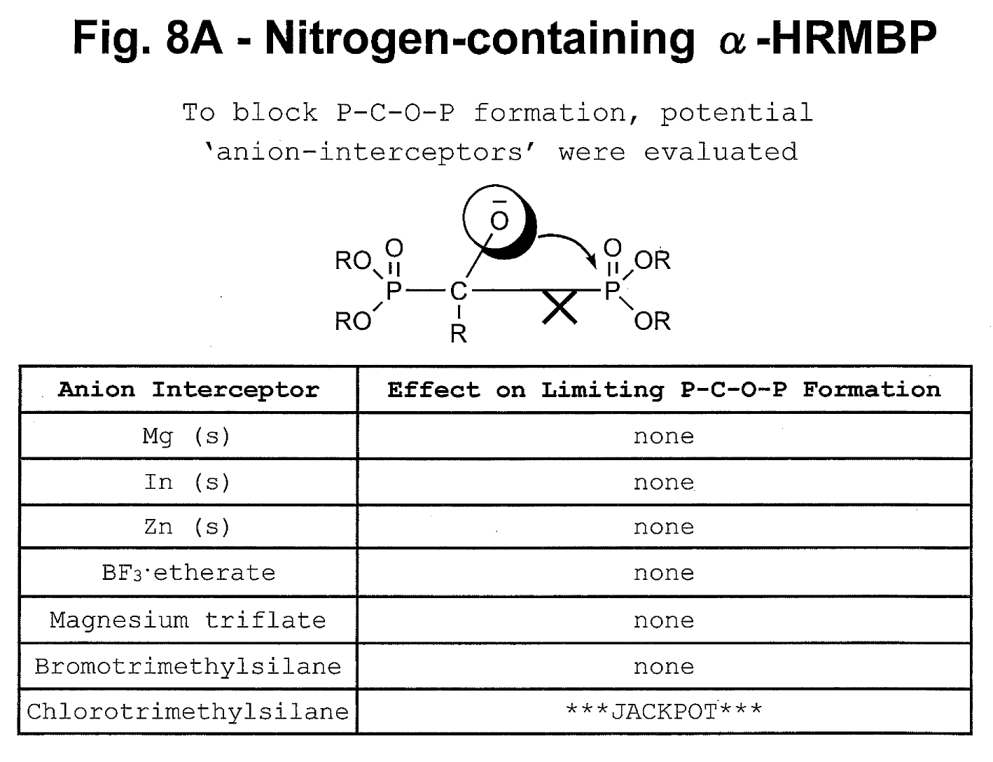

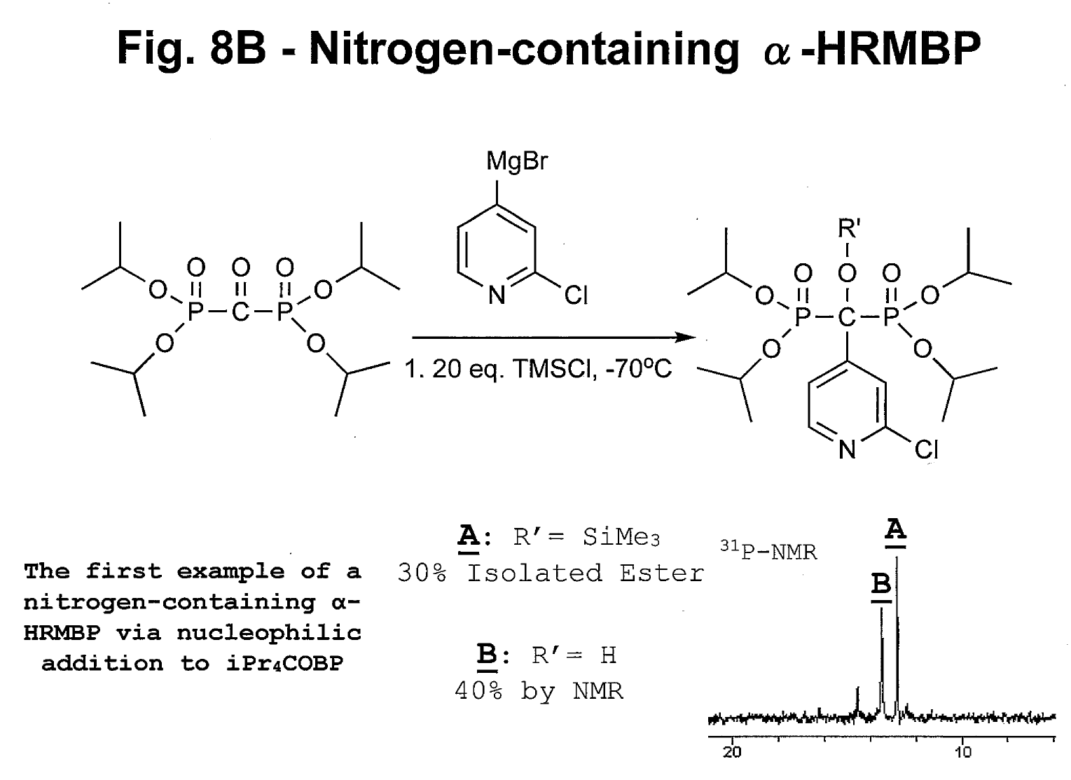

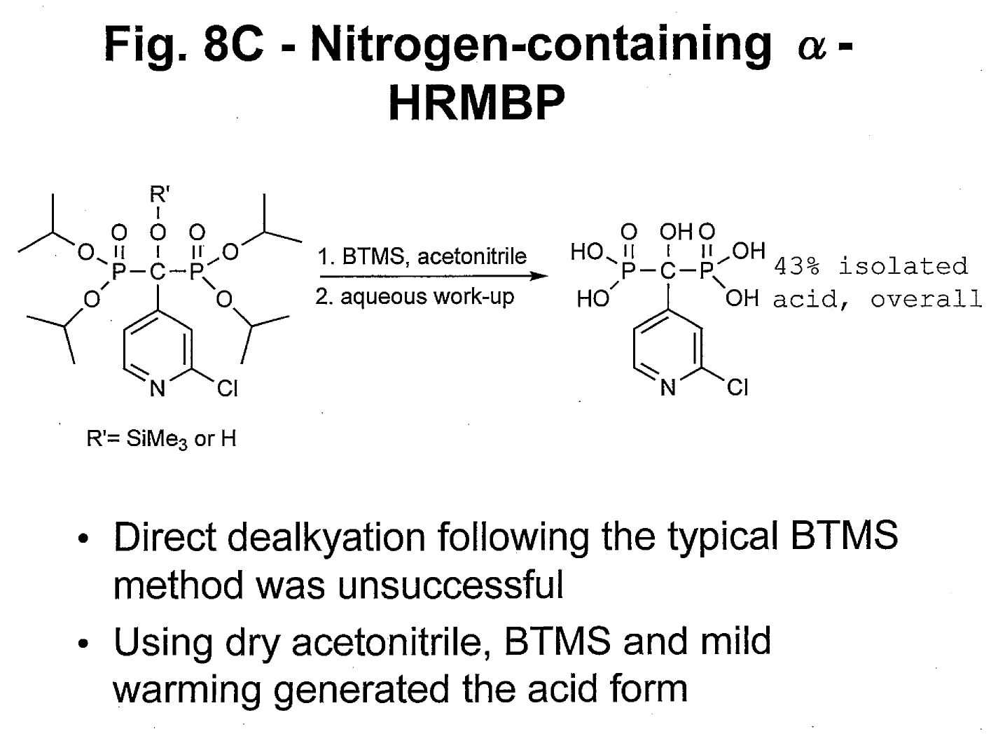

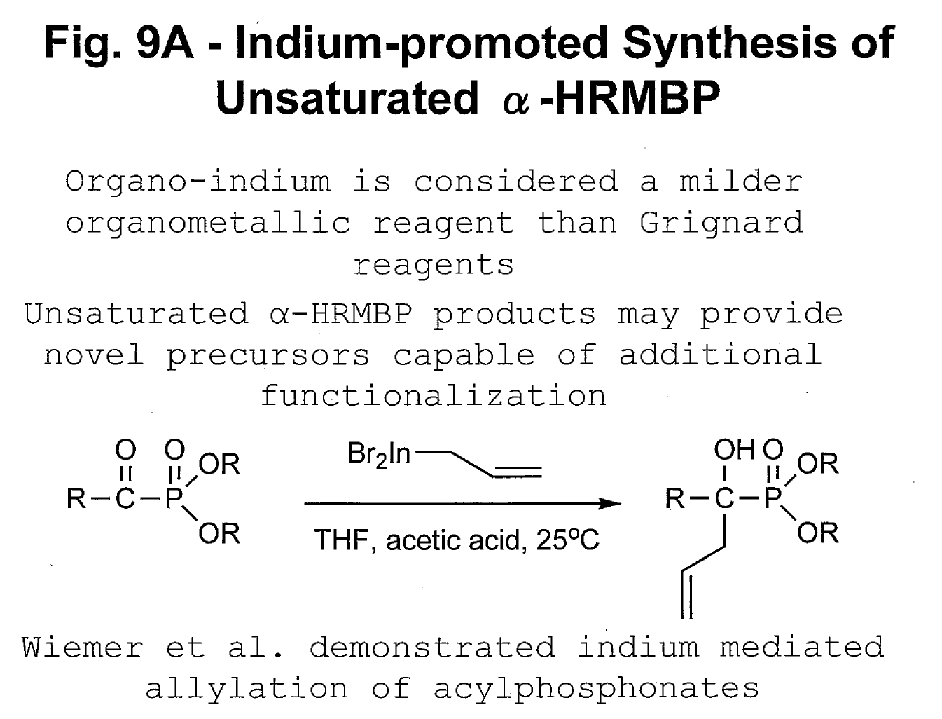

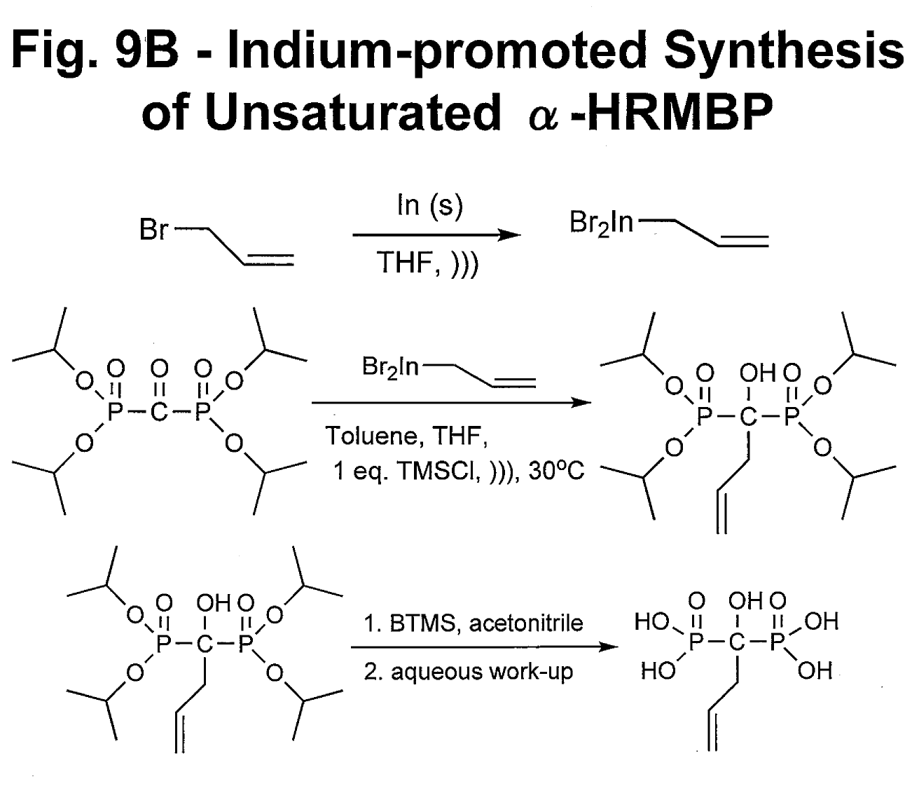

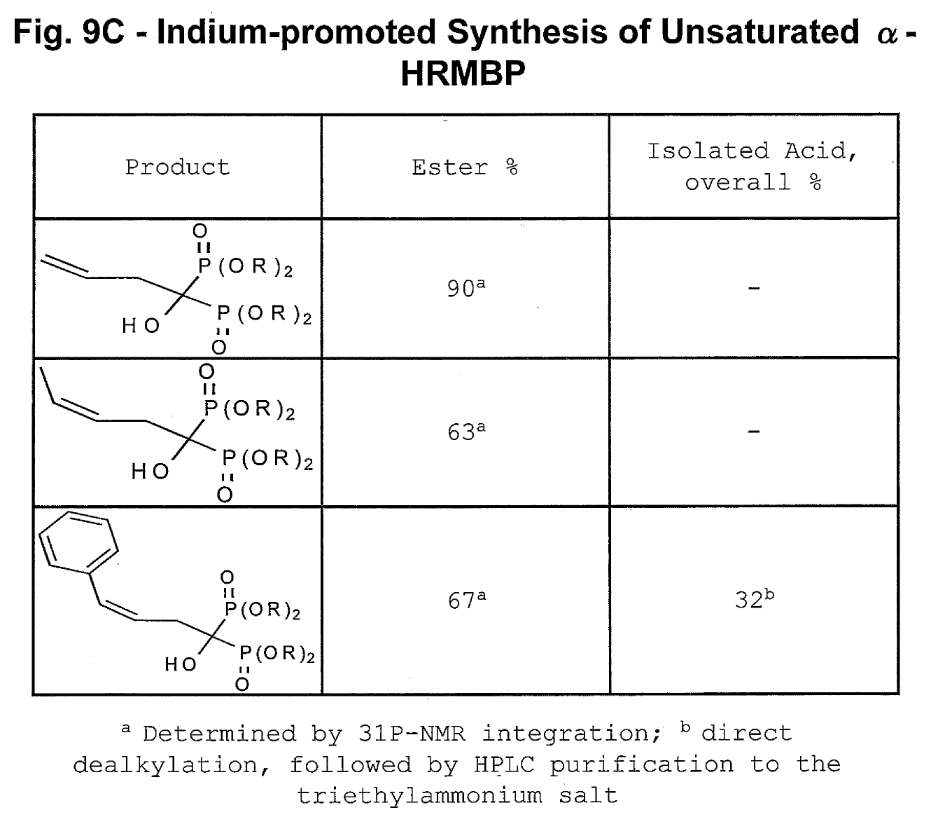

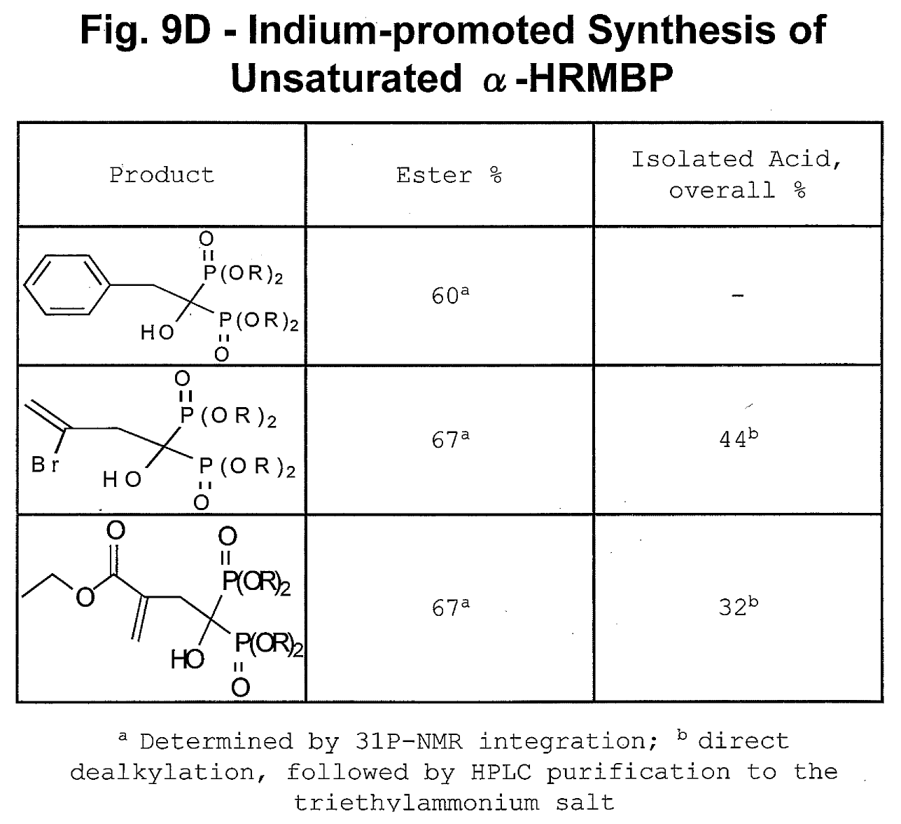

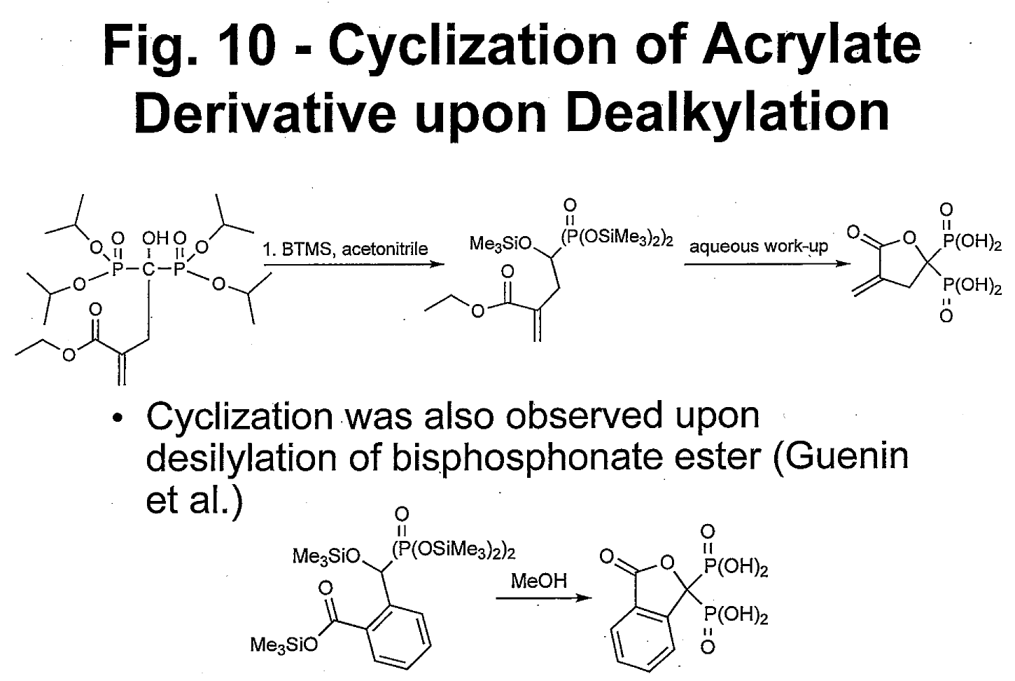

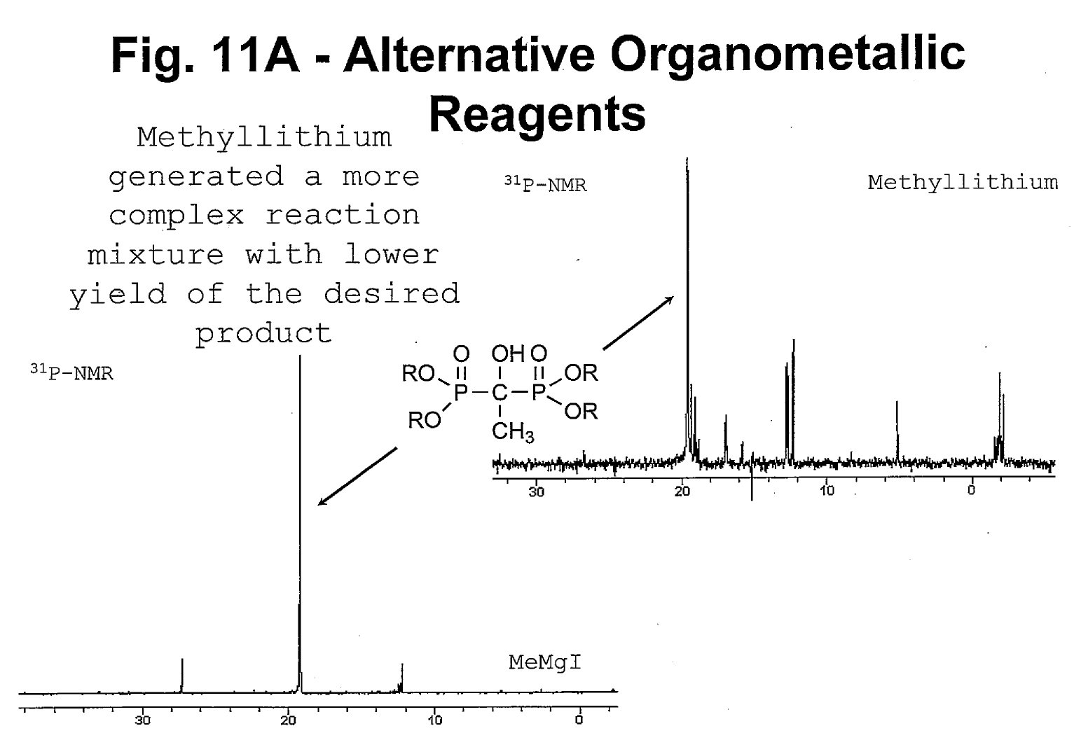

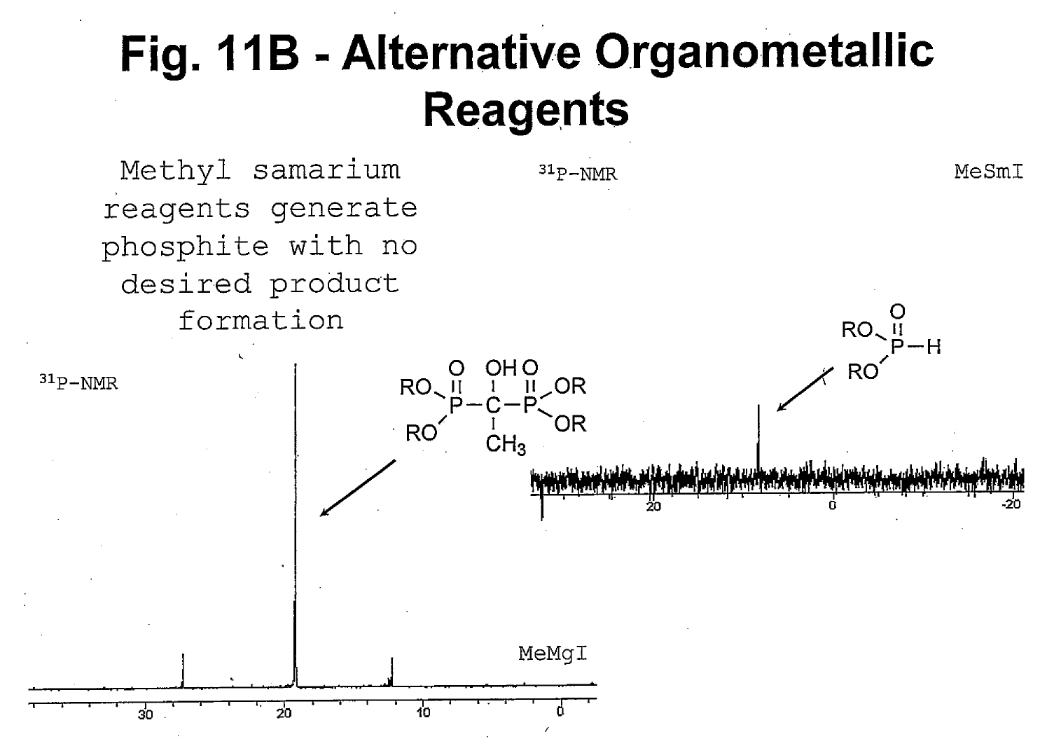

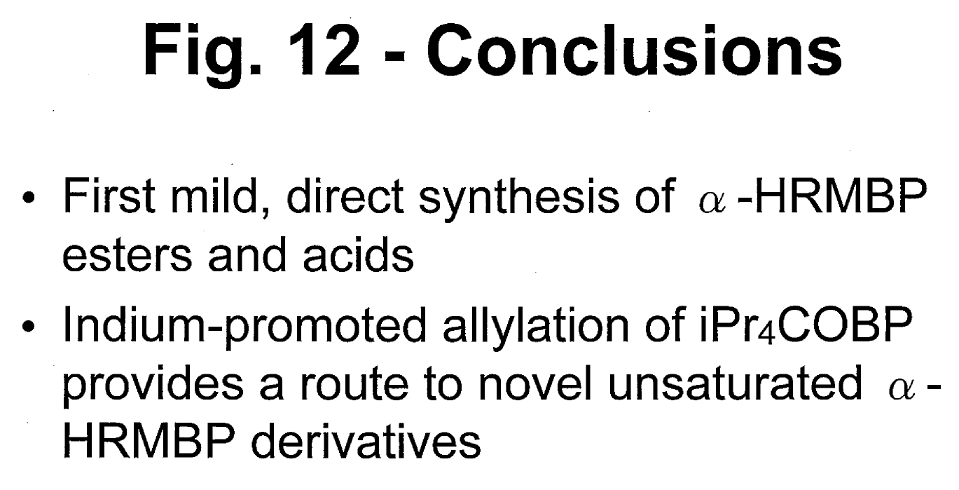

### DETAILED DESCRIPTION OF THE INVENTION

The present invention provides advancements to date in the generation of α-HRMBP and α-HRMPA acids and related derivatives from various organometallic reagents employing specific methods for limiting common rearrangement/decomposition pathways that lead to side products. NMR-scale reactions of alkyl and aryl Grignard reagents with iPr₄COBP^{28, 32} established precedence for the facile creation of product libraries.

More specifically, one method of the invention for preparing a compound comprises contacting iPr₄COBP with a Grignard reagent to form an alkyl or aryl substituted α-HRMBP, in the form of an ester. A Grignard reagent is a reagent obtained by reacting a suitable alkyl or aryl halide with Mg under conditions well known to those skilled in the art. In some embodiments, the Grignard reagent is referred to as RMgX, in which, R is an alkyl or aryl (e.g., methyl, phenyl, or benzyl) group and X is a halo group. The Grignard reagent of the invention is generated in Et₂O:THF. The ratio of Et₂O:THF is in the range of 5:1 to 1:5, preferably 4:1 to 1:4, 3:1 to 1:3, or 2:1 to 1:2, and most preferably is 1:1. The term “substituted” refers to moieties having one, two, three, or more substituents, which may be the same or different, each replacing a hydrogen atom.

For example, Scheme 1.5 illustrates optimized reactions for synthesis on a 0.5-1.0 g or other scale, improving the yields of 2b and 2c by 20% and 30% (by ³¹P NMR) respectively, via adjusting the Et₂O:THF ratio in the Grignard reagents to 1:1 before adding iPr₄COBP. The reaction product may be directly dealkylated under typical bromotrimethylsilane (BTMS) conditions^{33, 34}, purified by HPLC, and characterized as the triethylammonium salt.

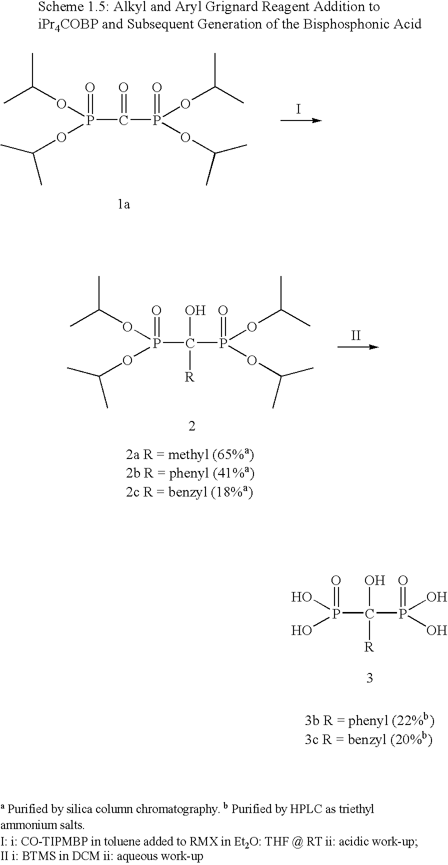

A second method of the invention for preparing a compound comprises contacting iPr₄COBP or COTEPA with a Grignard reagent and then an anion-interceptor to form an α-substituted α-HRMBP or α-HRMPA ester, which may be converted to an acid or salt using a method well known in the art or described in the present application.

The Grignard reagent may be a heterocyclic Grignard reagent, and the heterocyclic group may contain nitrogen. An example of a heterocyclic Grignard reagent is 2-chloropyridine Grignard reagent. Alternatively, the Grignard reagent may be any alkyl or aryl Grignard reagent. A heterocyclic, alkyl, or aryl Grignard reagent may be obtained from commercial vendors or synthesized using a method well known in the art or described in the present application.

The Grignard reagent may be generated in situ by a halogen exchange process. For example, Scheme 1.6 illustrates methods to synthesize Grignard reagents by an exchange reaction of isopropyl magnesium bromide with iodo compounds³⁶ that can be utilized in practicing the instant invention.

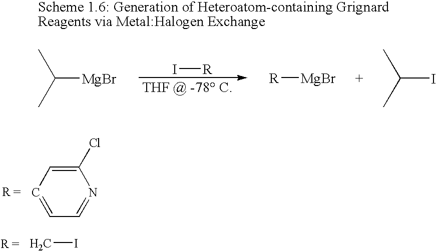

An anion-interceptor is a reagent that replaces the ionizable hydrogen in an OH group with a moiety that cannot be removed by a base, thus preventing formation of a reactive oxygen anion. In some embodiments, the anion-interceptor is a silylating agent such as CTMS, which converts the —OH group to an —OSi(CH₃)₃ group.

For example, Scheme 1.7 illustrates heteroatom-containing Grignard reagent addition to iPr₄COBP and subsequent generation of the bisphosphonic acid. Typical BTMS silyldealkylation^{33, 34} is ineffective for complete dealkylation of this product. Use of dried and distilled acetonitrile provides the dealkylated form of 6a and the 2-chloropyridine P—O—C—P derivative.

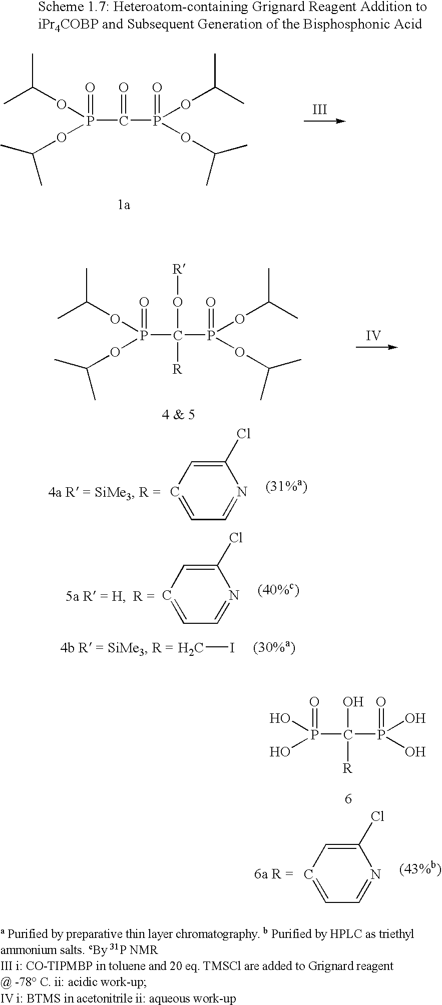

Scheme 1.8 illustrates heteroatom-containing Grignard reagent addition to COTEPA and subsequent generation of the phosphonoacetate. Silyldealkylation to the phosphonic acid is accomplished by BTMS affording the C-ethyl ester (8a). This may be converted to the triacid, if desired, by methods known in the art such as acid hydrolysis.

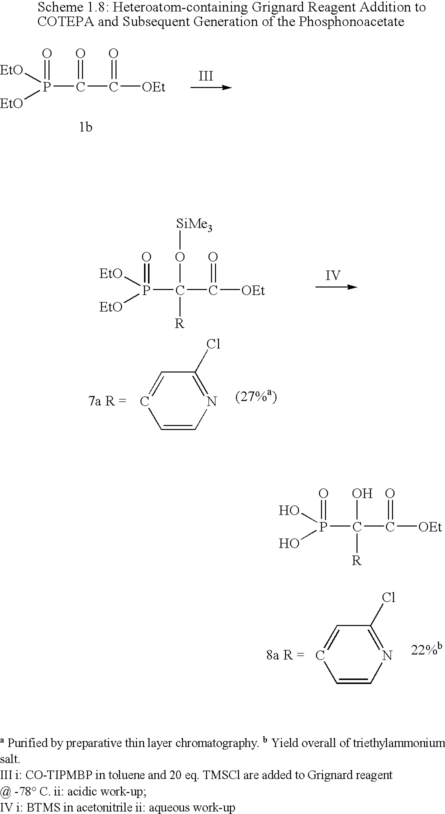

A third method of the invention for preparing a compound comprises contacting iPr₄COBP or COTEPA with an unsaturated indium halide reagent, such as allyl InBr₂, to form an unsaturated substituted α-HRMBP or α-HRMPA ester, which may be converted to an acid or salt using a method well known in the art or described in the present application. iPr₄COBP or COTEPA may be contacted with the unsaturated indium halide reagent in the presence of acetic acid or CTMS. Furthermore, iPr₄COBP or COTEPA may be contacted with the unsaturated indium halide reagent in the presence of a Lewis acid. A Lewis acid is a compound containing an atom capable of accepting a pair of electrons from a suitable donor molecule (Lewis base). A well-known example of a Lewis acid includes, but is not limited to, BF₃.

For example, Scheme 1.10 illustrates allyl indium reagent additions to iPr₄COBP and subsequent generation of the bisphosphonic acid. Direct silyldeakylation of the ester is completed using BTMS in acetonitrile to access the corresponding acid in good yield. It should be noted that in certain cases, further modification of the product may be observed. In particular, the α-methylene ester derivative 9f further forms 10c via intramolecular cyclization.

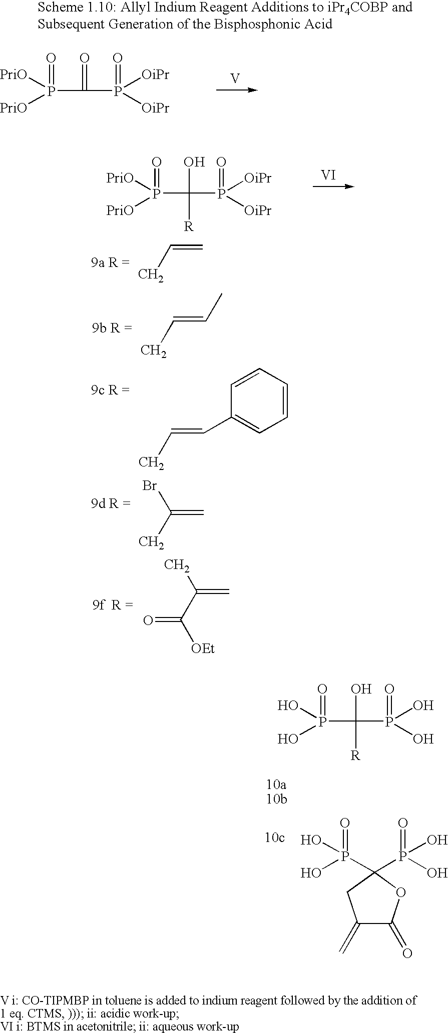

Scheme 1.11 illustrates allyl indium reagent additions to COTEPA and subsequent generation of the phosphonoacetate. Generation of the carboxylic acid form of 12b, product 12c, can be effected in various commonly known ways, including simply by prolonged exposure to the HPLC buffer used.

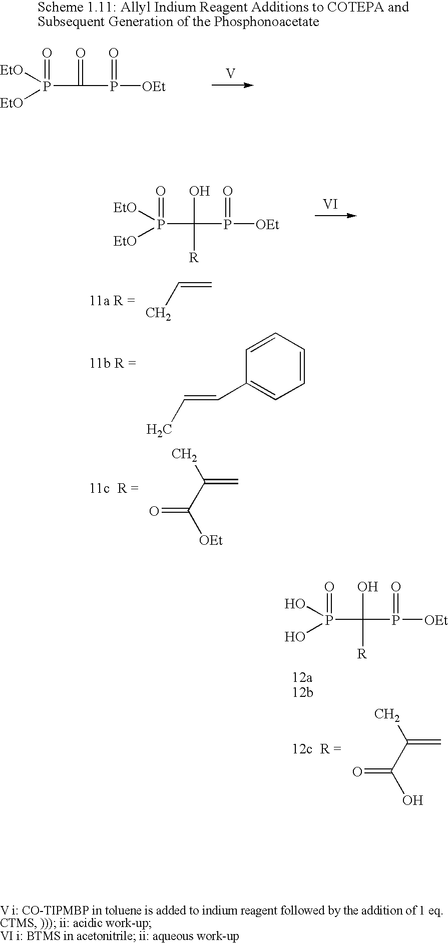

Furthermore, the invention provides the compounds prepared according to the methods described above. Such compounds include, but are not limited to, 4a, 5a, 6a, 9a-d and f, 10a-c, 11a-c, and 12a-c. As mentioned above, bisphosphonate and phosphonoacetate derivatives are useful for the treatment of bone diseases, cancer, and viral infection. The methods of the invention can be used to prepare such bisphosphonate and phosphonoacetate derivative drugs.

In particular, α-hydroxyl, unsaturated bisphosphonates find application for the preparation of dental materials such as self-etching primers or enamel-dentin adhesives. Currently used self-etching enamel-dentin adhesives are composed of methacrylate (or similar unsaturated functionalities such as acrylate or vinyl groups) containing phosphoric acids, which modify the enamel and dentin surface and mediate the formation of a strong bond to the composite restorative materials. However, these types of compounds are easily hydrolyzed. Thus, polymerizable bisphosphonates, such those containing methacrylate or other polymerizable moieties, will be much more stable to hydrolysis than their phosphoric acid counterparts. Conventional photopolymerization and/or chemical polymerization techniques could then be employed to convert the bisphosphonate monomer into the appropriate polymer for dental uses.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Results and Discussion

Early findings by Maeda et al. demonstrated that acylphosphonates could react with alkyl Grignard reagents to generate the stable dialkyl α-hydroxy, α-alkyl phosphonate, by adjusting solvents to limit fractionation²⁷. The inventors previously introduced a small-scale synthesis of 2a-c via nucleophilic addition of the respective Grignard reagents with iPr₄COBP^{28, 32}. The inventors have optimized these reactions for large-scale (0.5-1.0 g) synthesis, improving the yields of 2b and 2c by 20% and 30% (by ³¹P NMR) respectively, via adjusting Et₂O:THF ratios in the Grignard reagents before adding iPr₄COBP (Scheme 1.5). During this time, the inventors discovered that the tetraisopropyl esters of 2b and 2c could not be efficiently purified on a silica gel column due to an increase in rearrangement and fragmentation on the column. Furthermore, Ruel et al. observed that bulkier substituents were more sensitive to rearrangement²². Chromatography was circumvented by direct dealkylation of the reaction mixture under typical “McKenna reaction” bromotrimethylsilane (BTMS) conditions^{33, 34}, the product being purified by HPLC and characterized as the triethylammonium salt.

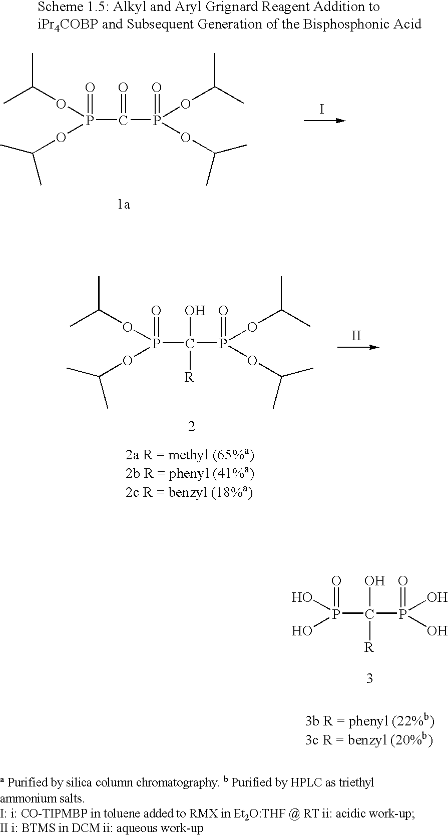

As nitrogen-containing heterocyclic α-HRMBP inhibitors are known to exhibit high potency in treating diseases involving abnormal bone metabolism, the inventors addressed the problem of generating a heterocyclic Grignard reagent for reaction with iPr₄COBP. There are several literature examples of how to synthesize functionalized heterocyclic Grignard reagents³⁵⁻³⁷. Knochel et al. described a method to synthesize heterocyclic Grignard reagents via a low temperature halogen-magnesium exchange³⁶. 2-Chloro-4-iodopyridine was chosen for facile generation of the corresponding Grignard reagent¹⁶ (Scheme 1.6).

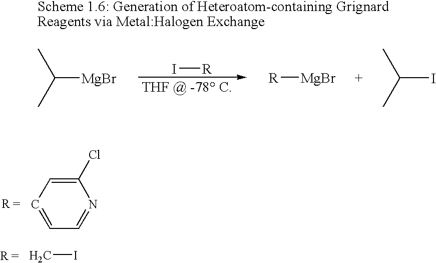

The inventors' first attempt at the synthesis of the 2-chloropyridine methylenebisphosphonate derivative produced P—O—C—P as the major product. Considering the mechanism of rearrangement, the inventors propounded the idea of intercepting the oxygen anion with a silylating agent such as chlorotrimethylsilane (CTMS). After several attempts, the inventors discovered that co-addition of iPr₄COBP with excess CTMS (20 eq.) at low temperatures (−78° C.) generated both 4a and 5a in a 3:2 ratio according to ³¹P NMR integration (Scheme 1.7). While not wanting to be bound by the theory, the inventors consider that excess CTMS immediately silates the oxygen anion producing HCl, which in turn protonates pyridine, blocking further base-catalyzed rearrangement. As observed with the aryl products, rearrangement and fragmentation during large-scale chromatography prompted the conversion of the crude ester to the corresponding acid. Typical BTMS dealkylation conditions^{33, 34} were found to be ineffective for complete dealkylation of this product. Use of dried and distilled acetonitrile as solvent provided the dealkylated form of 6a and the 2-chloropyridine P—O—C—P derivative.

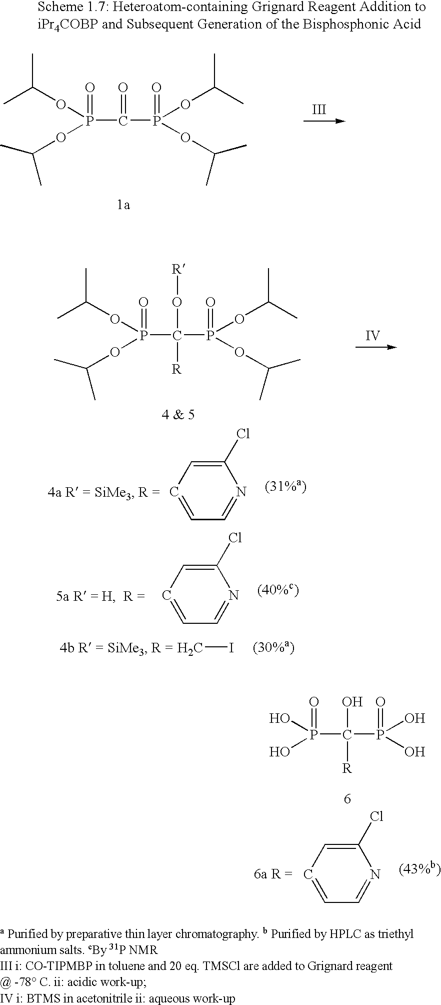

Employing this modification with the 2-chloropyridine Grignard addition to COTEPA successfully provides the silyl-protected α-HRMPA intermediate (7a), which proved to be less labile on silica gel in comparison to 4a. Dealkylation to the phosphonic acid was accomplished by BTMS affording the ethyl ester (8a) (Scheme 1.8).

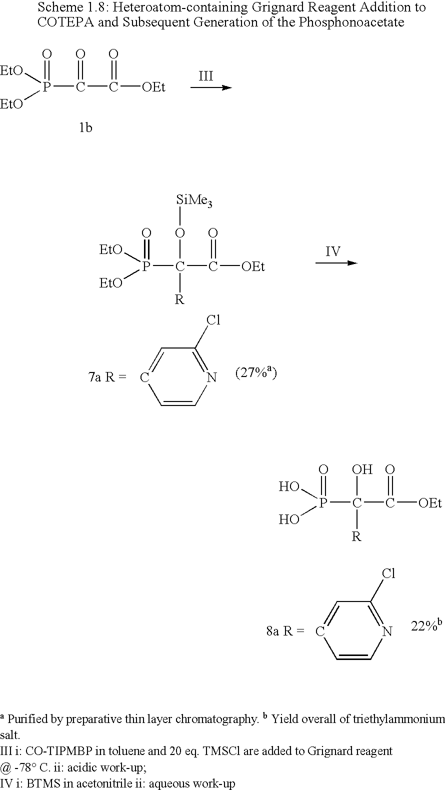

Directing their attention to the synthesis of the methylene-bridged heterocyclic derivatives, the inventors found the generation and use of heteroatom-containing ‘benzylic-like’ Grignard reagents challenging^{38, 39}. Exploration commenced on α-hydroxy-protected methylenephosphonates containing an α-methyl halide substituent, which could be later functionalized to the desired bridged heterocycle. Braun et al. demonstrated that diiodomethane undergoes halogen-magnesium exchange at low temperature, generating the corresponding Grignard reagent⁴⁰ (Scheme 1.7). Following the above procedure using the ‘CTMS-modification,’ 4b was generated and found to be stable as a yellow crystal if properly stored after silica gel chromatography (Scheme 1.7).

Work with Grignard reagents provided motivation to further study other organometallic reagents with iPr₄COBP and COTEPA. Alkyl lithium, samarium and magnesium-lithium reagents in addition to aryl zinc reagents were found to propagate rearrangement and decomposition chemistry with these two substrates, according to ³¹P NMR studies. Wiemer et al. demonstrated that allyl InBr₂ reagents react with acyl phosphonates to yield the corresponding α-hydroxy alkylphosphonate in the presence of acetic acid⁴¹. With respect to the other organometallic reagents studied, apart from zinc reagents, indium reagents are milder, exhibiting a higher tolerance for the presence of heteroatoms and are relatively unaffected by oxygen and water⁴².

Following Wiemer's protocol⁴¹, the inventors attempted the allyllation of iPr₄COBP using in situ-generated allyl InBr₂ in the presence of acetic acid. ³¹P NMR spectral analysis of the reaction mixture confirmed that no P—O—C—P formed; however, only a small amount of the desired product was seen, with a trimer as the major component. ³¹P NMR experiments showed that indium metal and acetic acid promote the decomposition of carbonylbisphosphonates to phosphite, resulting in the unwanted trimer formation. The role of acetic acid in these reactions was examined by comparing the effects of acids with varying acidities. Formic acid (pKa=3.75) and p-nitrophenol (pKa=7.2) were used to replace the acetic acid (pKa=4.7) but with no enhancement in product yield.

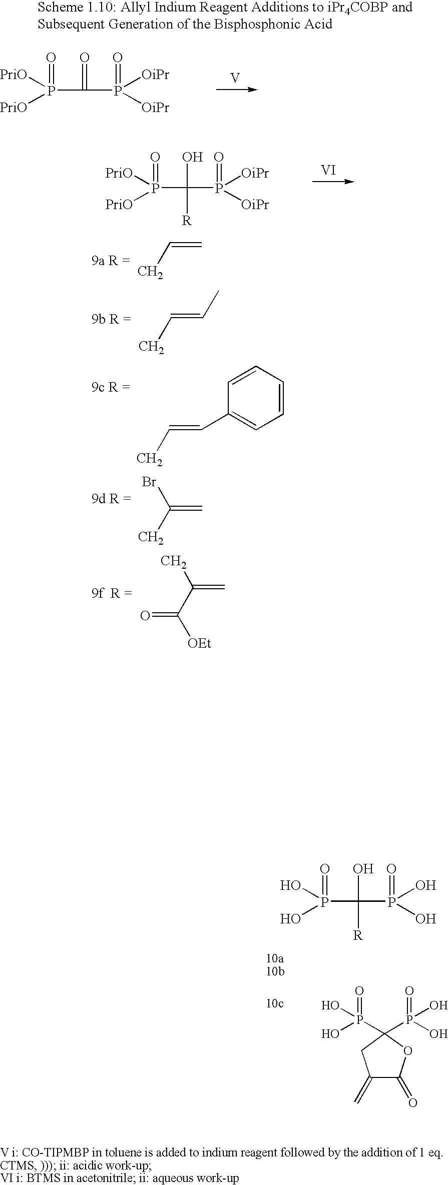

Lewis acids, such as BF₃.etherate, have been shown to enhance or catalyze organometallic reactions. Augé et al. observed that allylation of aldehydes and ketones may be enhanced by stoichiometric amounts of indium(s) and CTMS⁴³ but the relevance of this reaction to organophosphorus compounds, in particular bisphosphonates and phosphonoacetates, is not known. Surprisingly, a significant increase in the allylation product was observed when 1 eq. of CTMS was added replacing the acetic acid in the reaction mixture, immediately following the addition of CO-TIPMBP and sonication. Direct deakylation of the ester was completed using BTMS in acetonitrile to provide the corresponding acid in good yield. This improved protocol can be extended to synthesize other novel α-hydroxy, α-unsaturated BP and PA derivatives (Schemes 1.10 and 1.11).

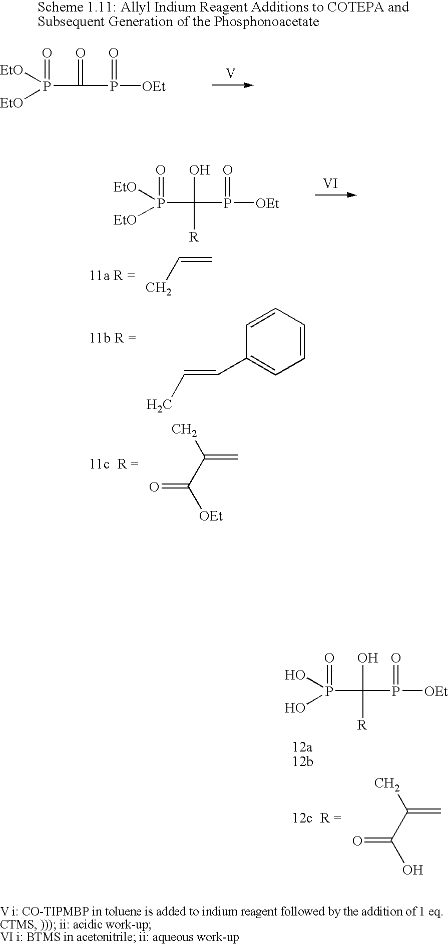

The α-methylene ester derivative 9f may undergo intramolecular cyclization. Lactone formation in compounds containing α-hydroxy, α-methylene esters under acidic conditions is not uncommon, especially when flanked by strong electron withdrawing groups (Scheme 1.12)^{44, 45}. The acidic conditions generated upon aqueous work-up of the BTMS reaction mixture promote cyclization of the bisphosphonate (9f) but not the phosphonoacetate derivative, which provides the ester (12b) as the predominant product. Hydrolysis to 12c was facilely achieved by hydrolytic cleavage after prolonged exposure to the HPLC mobile phase buffer.

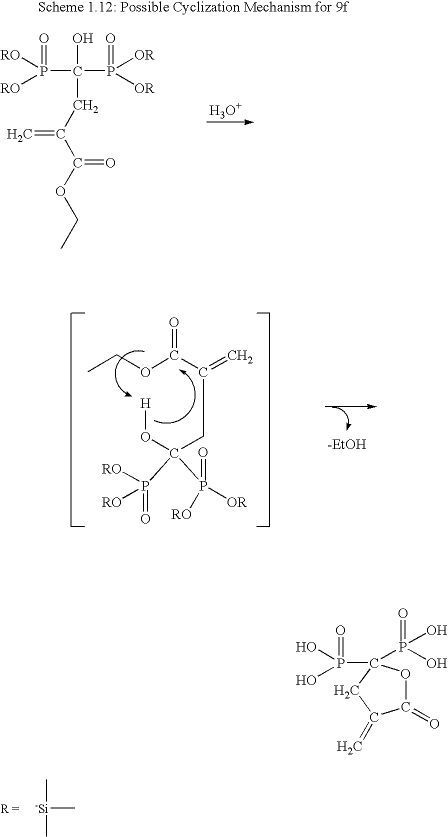

### Materials and Methods

All reactions were carried out under a nitrogen atmosphere, unless otherwise indicated. Toluene and tetrahydrofuran (THF) (both reagent grade purchased from Mallinckrodt Chemicals) were dried and distilled over sodiumibenzophenone. Ethyl acetate and acetonitrile (both HPLC grade purchased from Mallinckrodt Chemicals) were dried and distilled over P₂O₅. Anhydrous diethyl ether (Et₂O) was purchased from EMD Chemicals, Inc. Acetone (HPLC grade) was purchased from Mallinckrodt Chemicals and hexanes (reagent grade) from EM Science. Isopropylmagnesium bromide (15% in THF, ca. 1 mol/L) was purchased from TCI. Indium metal (100 mesh, 99.99%), magnesium turnings (≧99.5%), chlorotrimethylsilane (≧99%), bromotrimethylsilane (97%), and all halogenated starting materials were purchased from Sigma Aldrich. Tetraisopropyl methylenebisphosphonate and triethyl phosphonoacetate were graciously donated by Albright & Wilson Americas, Inc. iPr₄COBP and COTEPA were synthesized in situ via “the moisture modification” (McKenna et al.^{28, 31, 32}) and co-evaporated from ethyl acetate with dry toluene. Thin layer chromatography plastic-back sheets (20×20; silica gel 60 F₂₅₄) were purchased from EMD Chemicals, Inc. Preparative thin layer chromatography glass-back sheets (20×20; 1000 microns) were purchased from Analtech. Silica gel 150 (60-200 mesh) used for column chromatography (column width 1-2 in) of bisphosphonate esters was purchased from Mallinckrodt Chemicals; the esters were eluted using either a gradient from 100% toluene to 1:1 acetone:toluene or from 100% hexanes to 2:3 acetone:hexanes. Preparative HPLC was accomplished using the Waters 600E Multisolvent Delivery System with Waters 486 Tunable Absorbance Detector, equipped with a Varian Dynamax column (Microsorb 100-5, C₁₈; 250×21.4 mm). The mobile phase was 0.1 M triethylamine/acetic acid (0.1 M TEA:AA) at pH 7.0 using a gradient of 1% to 20% acetonitrile (HPLC grade) at a flow rate of 8 mL/min, detected at λ=254 nm.

Proton (¹H), carbon (¹³C), and phosphorus (³¹P) NMR spectra were measured either on a Bruker AM-360 MHz, Varian Mercury-400 MHz, or Bruker AMX-500 MHz spectrometer. Chemical shifts are reported relative to external TMS (¹H), internal CDCl₃ [δ=77.0] (¹³C) or external 85% H₃PO₄ (³¹P). NMR samples of BP and PA esters were dissolved in CDCl₃, while BP and PA triethylammonium salts were dissolved in D₂O. Triethylammonium salt peaks are on average 1.08 (t, ²J_{HH}=7, 3H); 3.00 (q, ²J_{HH}=7, 2H) for (¹H) and 7.8 (s, CH₃); 46.2 (s, CH₂) for (¹³C) and are omitted from the reported NMR spectral data. High-resolution mass spectrometry was performed at the University of California at Riverside High Resolution Mass Spectrometry Facility using a VG-ZAB mass spectrometry instrument, operated in the negative ion mode.

### Preparation of Substituted 1-hydroxymethylene-1-phosphonic acid, Alkyl Ester Derivatives via Grignard Chemistry

### Tetraisopropyl(1-hydroxyethane-1,1-diyl)bis(phosphonate) (2a)

The methyl Grignard reagent⁴⁶ (5 eq.) was obtained from magnesium turnings and methyliodide in 10 mL of a 1:1 dry Et₂O:THF solution at 5° C. iPr₄COBP (0.200 g, 0.54 mmol) was generated in situ and co-evaporated from ethyl acetate using 2 mL dry toluene and added via a glass syringe to a magnetically stirred solution of the Grignard reagent at 5° C. After 10-25 min, 5 mL of 10% acetic acid was added at 5° C. and stirred magnetically for 10 min. The aqueous phase was extracted twice with 5 mL portions of Et₂O. The organic layer and Et₂O extracts were combined and dried over Na₂SO₄, filtered and the solvent removed by rotary evaporation under reduced pressure at 50° C. The residue was purified by column chromatography eluted using a gradient from 100% toluene to 1:1 acetone:toluene. Solvent was removed by rotary evaporation under reduced pressure (˜1 mm Hg) at room temperature to constant weight, leaving 2a as a viscous oil (0.135 g, 65% yield overall).

δ_{P} 19.4⁴⁷

δ_{H} 1.3-1.4 (m, 24H), 1.6 (t, ³J_{HP}=16 Hz, 3H), 4.6-4.8 (m, 4H)⁴⁷

### Tetraisopropyl[Hydroxy(phenyl)methylene]bis(phosphonate) (2b)

Prepared as for 2a. 2b was obtained as a viscous yellow oil (0.10 g, 41% yield overall).

δ_{P} 15.4 (s)

δ_{H} 1.2 (m, 24H), 4.6 (m, 4H), 7.2-7.9 (m, 5H)

### Tetraisopropyl(1-Hydroxy-2-phenylethane-1,1-diyl)bis(phosphonate) (2c)

Prepared as for 2a. 2c was obtained as a light yellow viscous oil (0.045 g, 18% yield).

δ_{P} 19.6 (s)

δ_{H} 1.3 (m, 24H), 3.5 (t, ³J_{HP}=14 Hz, 2H), 4.6 (m, 4H), 7.2-7.9 (m, 5H)

### Tetraisopropyl[(2-chloropyridine-4-yl)[(1-trimethylsilyl)oxy]methylene]bis(phosphonate) (4a)

The Grignard reagent of 2-chloro-4-iodopyridine was synthesized according to Abarbri et al.¹⁶ In a 25 mL pear-shape flask, iPr₄COBP (0.200 g, 0.54 mmol) was generated in situ and co-evaporated from ethyl acetate using 5 mL toluene. CTMS (1 mL, ˜10 eq.) was added by a glass syringe under N₂ (g) and subsequently taken back up into the glass syringe. The ketone/CTMS solution was added to a magnetically stirred solution of the pyridinyl Grignard reagent (1.5 eq. in THF) at −60° C. (dry ice/acetone bath). The solution was first stirred at −60° C. for 20 min, then at room temperature for 10 min. The reaction mixture was worked up by adding 5 mL of 10% acetic acid at 5° C. stirring magnetically for 10 min. The aqueous phase was extracted twice with 5 mL portions of Et₂O. The organic layer and Et₂O extracts were combined and dried over Na₂SO₄, filtered and the solvent removed by rotary evaporation under reduced pressure. The residue was purified by preparative thin layer chromatography (silica gel) using a 1:1 acetone:toluene mobile phase (R_{f}=0.68). Solvent was removed by rotary evaporation under reduced pressure (˜1 mm Hg) at room temperature to a constant weight, leaving 4a as a light yellow viscous oil (0.091 g, 31% yield overall).

δ_{P} 13.1 (s)

δ_{H} 0.3 (s, 9H), 1.2-1.3 (m, 24H), 4.6 (m, 2H), 4.8 (m, 2H), 7.6 (broad m, 1H), 7.7 (broad s, 1H), 8.3 (broad d, ³J_{HH}=5, 1H)

δ_{C} 2.7 (s), 23.8 (m), 73.1 (d, ²J_{PC}=30 Hz), 121.1 (s), 122.8 (s), 148.3 (s), 150.8 (s)

### Ethyl(2-chloropyridin-4-yl)(diethoxyphosphoryl)[1-(trimethylsilyl)oxy]methylene]acetate (7a)

Prepared as for 4a using 0.135 g (0.54 mmol) of COTEPA. 7a was purified by preparative thin layer chromatography using a 2:3 acetone:hexane mobile phase, providing a light yellow viscous oil (0.062 g, 27% yield overall).

δ_{P} 13.2 (s)

δ_{H} 0.23 (s, 9H), 1.2 (m, 6H), 1.3 (t, ³J_{HH}=7, 3H), 4.1 (m, 4H), 4.3 (m, 2H), 7.5 (broad m, 1H), 7.6 (broad s, 1H), 8.3 (broad d, ³J_{HH}=5 Hz, 1H)

### Tetraisopropyl[1-[(trimethylsilyl)oxy]-2-iodoethane-1,1-diyl]bis(phosphonate) (4b)

The Grignard reagent of diiodomethane was synthesized according to Braun⁴⁰. 4b was prepared for as 4a. Purification was accomplished by preparative thin layer chromatography using a 1:1 acetone:toluene mobile phase (R_{f}=0.86) providing yellow crystals (0.091 g, 30% yield overall).

δ_{P} 12.3 (s)

δ_{H} 0.3 (S, 9H), 1.3 (m, 24H), 3.7 (t, ³J_{HP}=14, 2H), 4.8 (m, 4H)

δ_{C} −0.1 (s), 5.8 (s), 21.5 (m), 69.1 (s), 70.3 (s)

HR-MS (MH⁺): calcd. for 573.1063, found 573.1061

### Preparation of Substituted 1-hydroxymethylene-1-phosphonic acid triethylammonium salts

### [Hydroxy(phenyl)methylene]bis(phosphonic acid), triethylammonium salt (3b) from 2b

Direct dealkylation of the 2b reaction mixture (70% by ³¹P NMR) was performed by first drying the residue by rotary evaporation under reduced pressure (˜1 mm Hg) at room temperature to a constant weight. Neat BTMS (0.5 mL, ˜10 eq.) was added under N₂ (g) and the mixture was stirred magnetically for 12 hrs.

Unreacted BTMS was removed by rotary evaporation under reduced pressure at 40° C. To the residue, 5 mL of water was added. After being stirred magnetically for 30 min at room temperature, the mixture was extracted twice with 5 mL portions of Et₂O. The aqueous phase was collected and water removed by rotary evaporation under reduced pressure (˜1 mm Hg) at 40° C. leaving a light yellow viscous oil. The crude product was dissolved in 0.5 mL of 0.1 M TEA:AA at pH 7.0 and purified by HPLC using a linear gradient from 1% to 10% acetonitrile (t_{R}=16.8 min). Solvent was removed by rotary evaporation under reduced pressure (˜1 mm Hg) at 5° C. Excess triethylamine and acetic acid was removed by adding 0.5 mL of water and freeze-drying (˜0.7 mm Hg). This process was repeated twice, providing the pure salt of 3b as a white foam (0.054 g, 22% yield overall as a di-salt).

δ_{P} 15.5 (s)

δ_{H} 7.1-7.7 (m, 5H)

HR-MS (FAB⁻; [M²⁻+H⁺]⁻) calcd. for 266.9823, found 266.9816

### (1-Hydroxy-2-phenylethane-1,1-diyl)bis(phosphonic acid), triethylammonium salt (3c) from 2c

Prepared as for 3b. 3c was purified by HPLC using a 1% to 10% acetonitrile linear gradient (t_{R}=24.8 min), providing a white foam (0.051 g, 20% yield overall).

δ_{P} 17.5 (s)

δ_{H} 3.1 (t J_{HP}=14 Hz, 2H), 7.1-7.3 (m, 5H)

HR-MS (FAB⁻; [M²⁻+H⁺]⁻): calcd. for 280.9980, found 280.9986

### [(2-Chloropyridine-4-yl)-1-hydroxy-methylene]bis(phosphonic acid), triethylammonium salt (6a) from 4a

Direct dealkylation of the 4a reaction mixture (60% by ³¹P NMR) was employed. The crude product was dried under reduced pressure (˜1 mm Hg) at room temperature to a constant weight in a 25 mL pear-shape flask. Under N₂ (g) 4a was first dissolved with dry acetonitrile, followed by the addition of neat BTMS (0.5 mL, ˜10 eq.) and allowed to stir magnetically for 12 hours. Work up was similar to the procedure followed for 3b. 6a was purified by HPLC using a 1% to 10% acetonitrile linear gradient (t_{R}=19.3 min), providing a light yellow foam (0.116 g, 43% yield overall).

δ_{P} 12.9 (s)

δ_{H} 7.5 (broad m, 1H), 7.6 (broad s, 1H), 8.0 (d, ³J_{HH}=6, 1H)

δ_{C} 120.2 (s), 121.5 (s), 147.5 (s), 149.5 (s), 153.8 (s)

HR-MS (FAB⁻; [M²⁻+H⁺]⁻): calcd. for 301.9383, found 301.9384

### [1-(2-Chloropyridin-4-yl)-2-ethoxy-1-hydroxy-methylene]-2-oxoethyl]phosphonic acid, ethyl ester, triethylammonium salt (8a) from 7a

Prepared as for 6a (7a in the reaction mixture was 58% by ³¹P NMR). 8a was purified by HPLC using a 1% to 20% acetonitrile linear gradient (t_{R}=18.5 min), providing a light yellow foam (0.058 g, 22% yield overall as a mono-salt).

δ_{P} 13.1 (s)

δ_{H} 4.1 (m, 2H), 7.6 (broad m, 1H), 7.7 (broad s, 1H), 8.1 (broad d, ³J_{HH}=5 Hz, 1H)

HR-MS (FAB⁻; [M²⁻+H⁺]⁻): calcd. for 293.9937, found 293.9941

### Preparation of Unsaturated 1-hydroxymethylene-1-phosphonic acids, alkyl ester Derivatives via allyl InBr₂ Reagents

### Tetraisopropyl(4-hydroxybut-1-ene-4,4-diyl)bis(phosphonate) (9a)

Indium (s) (0.075 g, 0.65 mmol) and 5 mL THF were added to a 25 mL pear-shape flask. The flask was placed in an ultrasonicator (Bransonic 2510; 40 kHz) for 60 min at 30° C. Allyl bromide (0.055 mL, 0.65 mmol) was injected via a glass micro-syringe, and the flask was returned to the sonicator for an additional 60, min at 30-40° C. iPr₄COBP (0.200 g, 0.54 mmol) was generated in situ, co-evaporated from ethyl acetate with 3 mL of toluene and injected via a glass syringe into the indium reagent at 5° C. under N₂ (g). Immediately following the addition of ketone, 0.08 mL CTMS (0.634 mmol) was injected via a micro-glass syringe to the reaction mixture and further sonicated at 30-40° C. for 60-90 min or until the yellow color of the ketone was no longer observed. The reaction mixture was worked up by adding 5 mL of 10% acetic acid at 5° C. stirring magnetically for 5 min. The aqueous phase was extracted twice with 5 mL portions of Et₂O. The organic layer and Et₂O extracts were combined and dried over Na₂SO₄, filtered and the solvent removed by rotary evaporation under reduced pressure at 50° C. A small amount (0.020 g) of the crude product was purified by thin layer chromatography (using an iodine chamber to follow product movement) using a 1:1 acetone:toluene mobile phase. Some product decomposed on silica, decreasing the overall yield. Solvent was removed by rotary evaporation under reduced pressure (˜1 mm Hg) at room temperature to constant weight providing a colorless oil (90% yield by ³¹P NMR).

δ_{P} 18.6 (s)

δ_{H} 1.4-1.5 (m, 24H), 2.7 (dt, ³J_{HH}=4, ³J_{PH}=15, 2H), 4.7 (m, 4H), 5.0-5.1 (m, 2H), 6.1 (m, 1H)

### Tetraisopropyl[(2)-5-hydroxypent-2-ene-5,5-diyl)bis(phosphonate) (9b)

Prepared as for 9a. The product was not purified by chromatography (63% yield by ³¹P NMR), but was dried under reduced pressure (˜1 mm Hg) at room temperature to constant weight for direct dealkylation.

δ_{P} 18.1 (dd, ²J_{PP}=40 Hz)

### Tetraisopropyl[(1)-4-hydroxy-1-phenylbut-1-ene-4,4-diyl]bis(phosphonate) (9c)

Prepared as for 9a. The product was not purified by chromatography (67% yield by ³¹P NMR), but was dried under reduced pressure (˜1 mm Hg) at room temperature to constant weight for direct dealkylation.

δ_{P1} 16.2 (d, ²J_{PP}=37 Hz)

δ_{P2} 18.1 (d, ²J_{PP}=34 Hz)

### Tetraisopropyl(2-Bromo-4-hydroxybut-1-ene-4,4-diyl)bis(phosphonate) (9d)

Prepared as for 9a. The product was not purified by chromatography (67% yield by ³¹P NMR), but was dried under reduced pressure (˜1 mm Hg) at room temperature to constant weight for direct dealkylation.

δ_{P} 17.2 (s)

### Tetraisopropyl[2-(ethoxycarbonyl)-4-hydroxybut-1-ene-4,4-diyl]bis(phosphonate) (9f)

Prepared as for 9a. The product was not purified by chromatography (67% yield by ³¹P NMR), but was dried under reduced pressure (˜1 mm Hg) at room temperature to constant weight for direct dealkylation.

δ_{P} 16.9 (s)

### Ethyl 2-(diethoxyphosphoryl)-2-hydroxypent-4-enoate (11a)

Prepared as for 9a. The product was not purified by chromatography (55% yield by ³¹P NMR).

δ_{P} 14.7 (s)

### Diethyl 2-(diethoxyphosphoryl)-2-hydroxy-4-methylenepentanedioate (11b)

Prepared as for 9a using 0.162 g of COTEPA generated in situ and co-evaporated from ethyl acetate using 1.5 mL toluene. Product was purified by preparative thin layer chromatography using a 2:3 acetone:hexane mobile phase providing a light yellow oil (0.096 g, 42% yield overall).

δ_{P} 15.6 (s)

δ_{H} 1.1 (broad m, 6H), 1.4 (broad t, ³J_{HH}=7, 3H), 3.5 (m, 1H), 3.8 (m, 1H), 3.9 (m, 2H), 4.1 (d, ³J_{PH}=10, 1H), 4.3 (m, 2H), 5.1 (m, 2H), 6.1 (dt, ³J_{HH}=18, ³J_{PH}=10, 1H), 7.2-7.5 (m, 5H)

### 4-(Ethoxycarbonyl)-2-hydroxy-2-phosphonopent-4-enoic acid (11c)

Prepared as for 9a using 0.21 g of COTEPA generated in situ and co-evaporated from ethyl acetate using 1.5 mL toluene. The product was purified by preparative thin layer chromatography using a 2:3 acetone:hexane mobile phase providing a light yellow oil (0.089 g, 30% yield overall).

δ_{P} 14.5 (s)

δ_{H} 1.3 (m, 12H), 3.0 (dd, ²J_{HH}=14, ³J_{PH}=6, 1H), 3.2 (dd, ²J_{HH}=14, ³J_{PH}=9, 1H), 3.9 (d, ³J_{PH}=7, 1H), 4.2 (m, 6H), 4.3 (d, ³J_{PH}=7, 1H), 5.7 (s, 1H), 6.2 (s, 1H)

### Preparation of Unsaturated 1-hydroxymethylene-1-phosphonic acids, triethyl ammonium salts

### [(1)-4-Hydroxy-1-phenylbut-1-ene-4,4-diyl]bis(phosphonic acid), triethylammonium salt (10a) from 9c

Prepared as for 6a. 10a was purified by HPLC using a 1% to 10% acetonitrile linear gradient (t_{R}=26.3 min), providing a colorless oil (0.088 g, 32% yield overall).

δ_{P} 16.7 (s)

δ_{H} 3.8 (broad s, 1H), 4.7-4.9 (m, 2H), 6.3 (m, 1H), 7.0 (t, 3J_{HH}=7, 1H), 7.1 (t, ³J_{HH}=7, 2H), 7.2(d, ³J_{HH}=7, 2H)

δ_{C} 52.2 (s), 114.8 (s), 125.5 (s), 127.0 (s), 128.8 (s), 137.8 (s), 140.5 (s)

HR-MS (FAB⁻; M⁻): calcd. for 307.0136, found 307.0139

### (2-Bromo-4-hydroxybut-1-ene-4,4-diyl)bis(phosphonic acid), triethylammonium salt (10b) from 9d

Prepared as for 6a. 10b was purified by HPLC using a 1% to 10% acetonitrile linear gradient (t_{R}=24.8 min), providing a white foam (0.093 g, 34% yield overall).

δ_{P} 16.4 (s)

δ_{H} δ 5.5 (s, 1H), 5.7 (s, 1H)

HR-MS (FAB⁻; [M²⁻+H⁺]⁻): calcd. for 308.8928, found 308.8932

### (4-Methylene-5-oxotetrahydrofuran-2,2-diyl)bis(phosphonic acid), triethylammonium salt (10c) from 9f

Prepared according to the procedure for 6a. 10c was purified by HPLC using a 1% to 10% acetonitrile linear gradient (t_{R}=18.2 min), providing a colorless oil (0.091 g, 37% yield overall).

δ_{P} 12.9 (s)

δ_{H} 3.2 (t, ³J_{PH}=15, 2H), 5.7 (s, 1H), 6.0 (s, 1H)

δ_{C} 22.3 (s), 80.1 (t, ¹J_{PC}=108 Hz), 123.1 (s), 133.4 (s), 173.0 (s)

HR-MS (FAB⁻; M⁻): calcd. for 256.9616, found 256.9619

### [(3)-1-(Ethoxycarbonyl)-1-hydroxy-4-phenylbut-3-en-1-yl]phosphonic acid, triethylammonium salt (12a) from 11b

Prepared as for 6a. 12a was purified by HPLC using a 1% to 20% acetonitrile linear gradient (t_{R}=18.4 min), providing a white foam (0.09 g, 34% yield overall).

δ_{P} 12.2 (s)

δ_{H} 1.2 (t, ³J_{HH}=6, 3H), 4.0 (d, ³J_{PH}=9, 1H), 4.1 (m, 2H), 5.1 (m, 2H), 6.1 (dt, ³J_{HH}=18, ³J_{PH}=9, 1H), 7.1 (t, ³J_{HH}=7, 1H), 7.2 (t, ³J_{HH}=7, 2H), 7.3 (d, ³J_{HH}=7, 2H)

δ_{C} 14.1 (s), 55.6 (s), 62.3 (s), 80.9 (d, ¹J_{PC}=120 Hz), 117.5 (s), 126.5 (s), 127.2 (s), 128.4 (s), 136.1 (s), 138.9 (s), 174.3 (s)

HR-MS (M-H⁺): calcd. for 299.0684, found 299.0678

### [1,3-Bis(ethoxycarbonyl)-1-hydroxybut-3-en-1-yl]phosphonic acid, triethylammonium salt (12b) from 11c

Prepared as for 6a. 12b was purified by HPLC using a 1% to 20% acetonitrile linear gradient (t_{R}=25.8 min), providing a colorless oil (0.130 g, 39% yield overall).

δ_{P} 14.0 (s)

δ_{H} 2.8 (dd, ³J_{PH}=10, 1H), 4.0 (m, 4H), 5.6 (s, 1H), 6.0 (s, 1H)

HR-MS (M-H⁺): calcd. for 295.0582, found 295.0580

### 5-Ethoxy-4-hydroxy-2-methylene-5-oxo-4-phosphonopentanoic acid, triethylammonium salt (12c) from 11c

Prepared as for 6a. 12c was purified by HPLC using a 1% to 20% acetonitrile linear gradient (t_{R}=18.5 min), providing a colorless oil.

δ_{P} 10.1 (broad)

δ_{H} 3.3 (t, ³J_{PH}=14, 2H), 4.2 (m, 2H), 5.8 (s, 1H), 6.1 (s, 1H)

δ_{C} 13.0 (s), 34.6 (s), 63.2 (s), 124.3 (s), 133.8 (s), 171.3 (d), 173.2 (s)

HR-MS (M⁻): calcd. for 249.0164, found 249.0159

### REFERENCES

1. Fleisch, H. Bisphosphonates in Bone Disease: From the Laboratory to the Patient, Fourth Edition. Academic Press, St. Louis, 2000; 350 pp.

2. Kleerekoper, M.; Editor. Drug Therapy for Osteoporosis. 2005; 116 pp.

3. Lipton, A. Bisphosphonate therapy for patients with breast cancer. Current Cancer Therapy Reviews 2005, 1, (3), 217-225.

4. Berardinelli, F.; Iannucci, M.; Verratti, V.; Fusco, W.; Nicolai, M.; Tenaglia, R. L. Bisphosphonates treatment in metastatic prostate cancer. European Journal of Inflammation 2005, 3, (2), 49-54.

5. Daikoku, T.; Kudoh, A.; Fujita, M.; Sugaya, Y.; Isomura, H.; Shirata, N.; Tsurumi, T. Architecture of replication compartments formed during Epstein-Barr virus lytic replication. Journal of Virology 2005, 79, (6), 3409-3418.

6. Priestman, M. A.; Healy, M. L.; Becker, A.; Alberg, D. G.; Bartlett, P. A.; Lushington, G. H.; Schoenbrunn, E. Interaction of Phosphonate Analogues of the Tetrahedral Reaction Intermediate with 5-Enolpyruvylshikimate-3-phosphate Synthase in Atomic Detail. Biochemistry 2005, 44, (9), 3241-3248.

7. Li, L.; Murphy, K. M.; Kanevets, U.; Reha-Krantz, L. J. Sensitivity to phosphonoacetic acid: A new phenotype to probe DNA polymerase d in Saccharomyces cerevisiae. Genetics 2005, 170, (2), 569-580.

8. Reszka, A. A.; Rodan, G. A. Nitrogen-containing bisphosphonate mechanism of action. Mini-Reviews in Medicinal Chemistry 2004, 4, (7), 711-719.

9. Reszka, A. A.; Rodan, G. A. The mechanism of action of nitrogen-containing bisphosphonates. Osteoporosis 2003, 447-457.

10. Ebetino, F. H.; Roze, C. N.; McKenna, C. E.; Barnett, B. L.; Dunford, J. E.; Russell, R. G. G.; Mieling, G. E.; Rogers, M. J. Molecular interactions of nitrogen-containing bisphosphonates within farnesyl diphosphate synthase. Journal of Organometallic Chemistry 2005, 690, (10), 2679-2687.

11. Coxon, F. P.; Ebetino, F. H.; Mules, E. H.; Seabra, M. C.; McKenna, C. E.; Rogers, M. J. Phosphonocarboxylate inhibitors of Rab geranylgeranyl transferase disrupt the prenylation and membrane localization of Rab proteins in osteoclasts in vitro and in vivo. Bone (San Diego, Calif., United States) 2005, 37, (3), 349-358.

12. Mikroyannidis, J. A. Hydroxy- and/or carboxy-substituted phosphonic and bisphosphonic acids usable as corrosion and scale inhibitors. Phosphorus and Sulfur and the Related Elements 1987, 32, (3-4), 113-18.

13. Lazzarato, L.; Rolando, B.; Lolli, M. L.; Tron, G. C.; Fruttero, R.; Gasco, A.; Deleide, G.; Guenther, H. L. Synthesis of NO-Donor Bisphosphonates and Their in-Vitro Action on Bone Resorption. Journal of Medicinal Chemistry 2005, 48, (5), 1322-1329.

14. Xie, Y.; Ding, H.; Qian, L.; Yan, X.; Yang, C.; Xie, Y. Synthesis and biological evaluation of novel bisphosphonates with dual activities on bone in vitro. Bioorganic & Medicinal Chemistry Letters 2005, 15, (13), 3267-3270.

15. Neves, M.; Gano, L.; Pereira, N.; Costa, M. C.; Costa, M. R.; Chandia, M.; Rosado, M.; Fausto, R. Synthesis, characterization and biodistribution of bisphosphonates Sm-153 complexes: correlation with molecular modeling interaction studies. Nuclear Medicine and Biology 2002, 29, (3), 329-338.

16. Abarbri, M.; Thibonnet, J.; Berillon, L.; Dehmel, F.; Rottlaender, M.; Knochel, P. Preparation of New Polyfunctional Magnesiated Heterocycles Using a Chlorine-, Bromine-, or Iodine-Magnesium Exchange. Journal of Organic Chemistry 2000, 65, (15), 4618-4634.

17. Wei, C.; Lu, H.; Liang, B.; Wang, S.; Wang, S.; Shi, X.; Zhang, C.; Ma, G. Process for preparing 1-hydroxyl-1-carboxyalkylphosphonic acids. 2000-1289171309128, 20000915, 2001.

18. Lecouvey, M.; Leroux, Y. Synthesis of 1-hydroxy-1,1-bisphosphonates. Heteroatom Chemistry 2000, 11, (7), 556-561.

19. Lecouvey, M.; Mallard, I.; Bailly, T.; Burgada, R.; Leroux, Y. A mild and efficient one-pot synthesis of 1-hydroxymethylene-1,1-bisphosphonic acids. Preparation of new tripod ligands. Tetrahedron Letters 2001, 42, (48), 8475-8478.

20. Arstad, E.; Hoff, P.; Skattebol, L.; Skretting, A.; Breistol, K. Studies on the synthesis and biological properties of non-carrier-added [(125)I and (131)I]-labeled arylalkylidenebisphosphonates: potent bone-seekers for diagnosis and therapy of malignant osseous lesions. Journal of medicinal chemistry 2003, 46, (14), 3021-32.

21. Fitch, S. J.; Moedritzer, K. Nuclear magnetic resonance study of the P—C(OH)—P to P—CO—P rearrangement: Tetraethyl-I-hydroxyalkylidenediphosphonates. Journal of the American Chemical Society 1962, 84, 1876-9.

22. Ruel, R.; Bouvier, J.-P.; Young, R. N. Single-Step Preparation of 1-Hydroxybisphosphonates via Addition of Dialkyl Phosphite Potassium Anions to Acid Chlorides. Journal of Organic Chemistry 1995, 60, (16), 5209-13.

23. Burgos-Lepley, C. E.; Mizsak, S. A.; Nugent, R. A.; Johnson, R. A. Tetraalkyl oxiranylidenebis(phosphonates). Synthesis and reactions with nucleophiles. Journal of Organic Chemistry 1993, 58, (15), 4159-61.

24. Katzhendler, J.; Ringel, I.; Karaman, R.; Zaher, H.; Breuer, E. Acylphosphonate hemiketals—formation rate and equilibrium. The electron-withdrawing effect of dimethoxyphosphinyl group. Journal of the Chemical Society, Perkin Transactions 2: Physical Organic Chemistry 1997, (2), 341-349.

25. Pudovik, A. N.; Zimin, M. G. Addition reactions of partially-esterified phosphorus acids. Rearrangements of α-hydroxyalkyl phosphorus esters and their α-mercapto and α-amino-analogs. Pure and Applied Chemistry 1980, 52, (4), 989-1011.

26. Arstad, E.; Skattebol, L. Reactions of diethyl mesyl- or tosyloxyphosphonates with diethyl phosphite and base: a method claimed to yield bisphosphonates. Tetrahedron Letters 2002, 43, (48), 8711-8712.

27. Maeda, H.; Takahashi, K.; Ohmori, H. Reactions of acyl tributylphosphonium chlorides and dialkyl acylphosphonates with Grignard and organolithium reagents. Tetrahedron 1998, 54, (40), 12233-12242.

28. McKenna, C. E.; Kashemirov, B. A. Preparation and use of α-keto bisphosphonates. 99-US1577 2000002889, 19990713, 2000.

29. McKenna, C. E.; Kashemirov, B. A.; Roze, C. N. Carbonylbisphosphonate and (diazomethylene)bisphosphonate analogs of AZT 5′-diphosphate. Bioorganic Chemistry 2002, 30, (6), 383-395.

30. Bonaz-Krause, P. I.; Kashemirov, B. A.; McKenna, C. E. Oxidative pathways of α-diazo phosphonates. Phosphorus, Sulfur and Silicon and the Related Elements 2002, 177, (10), 2271.

31. Kashemirov, B. A.; Roze, C. N.; McKenna, C. E. Carbonylbisphosphonate analogues of nucleoside 5′-diphosphates. Phosphorus, Sulfur and Silicon and the Related Elements 2002, 177, (10), 2275.

32. McKenna, C. E.; Kashemirov, B. A. Recent progress in carbonylphosphonate chemistry. Topics in Current Chemistry 2002, 220, (New Aspects in Phosphorus Chemistry I), 201-238.

33. McKenna, C. E.; Higa, M. T.; Cheung, N. H.; McKenna, M. C. The facile dealkylation of phosphonic acid dialkyl esters by bromotrimethylsilane. Tetrahedron Letters 1977, (2), 155-8.

34. McKenna, C. E.; Schmidhauser, J. Functional selectivity in phosphonate ester dealkylation with bromotrimethylsilane. Journal of the Chemical Society, Chemical Communications 1979, (17), 739.

35. Trecourt, F.; Breton, G.; Bonnet, V.; Mongin, F.; Marsais, F.; Queguiner, G. New syntheses of substituted pyridines via bromine-magnesium exchange. Tetrahedron 2000, 56, (10), 1349-1360.

36. Knochel, P.; Diohle, W.; Gommermann, N.; Kneisel, F. F.; Kopp, F.; Korn, T.; Sapountzis, I.; Vu, V. A. Highly functionalized organomagnesium reagents prepared through halogen-metal exchange. Angewandte Chemie, International Edition 2003, 42, (36), 4302-4320.

37. Rottlander, M.; Boymond, L.; Berillon, L.; Lepretre, A.; Varchi, G.; Avolio, S.; Laaziri, H.; Queguiner, G.; Ricci, A.; Cahiez, G.; Knochel, P. New polyfunctional magnesium reagents for organic synthesis. Chemistry—A European Journal 2000, 6, (5), 767-770.

38. Beumel, O. F., Jr.; Smith, W. N.; Rybalka, B. Preparation of 2- and 4-picolyllithium. Synthesis 1974, (1), 43-5.

39. Sanchez-Sancho, F.; Herradon, B. Stereoselective conjugate addition of metalated 2-methylpyridine to functionalized α,β-unsaturated carbonyl compounds. Heterocycles 2003, 60, (8), 1843-1854.

40. Braun, H. A.; Meusinger, R.; Schmidt, B. 2-Iodoethanols from aldehydes, diiodomethane, and isopropylmagnesium chloride. Tetrahedron Letters 2005, 46, (15), 2551-2554.

41. Kim, D. Y.; Wiemer, D. F. Addition of allylindium reagents to acyl phosphonates: synthesis of tertiary α-hydroxy alkylphosphonates. Tetrahedron Letters 2003, 44, (14), 2803-2805.

42. Ranu, B. C. Indium metal and its halides in organic synthesis. European Journal of Organic Chemistry 2000, (13), 2347-2356.

43. Auge, J.; Lubin-Germain, N.; Marque, S.; Seghrouchni, L. Indium-catalyzed Barbier allylation reaction. Journal of Organometallic Chemistry 2003, 679, (1), 79-83.

44. Guenin, E.; Degache, E.; Liquier, J.; Lecouvey, M. Synthesis of 1-hydroxymethylene-1,1-bis(phosphonic acids) from acid anhydrides: Preparation of a new cyclic 1-acyloxymethylene-1,1-bisphosphonic acid). European Journal of Organic Chemistry 2004, (14), 2983-2987.

45. Steurer, S.; Podlech, J. Aminoalkyl-substituted a-methylene-g-butyrolactones from α-amino acids using an indium-mediated Barbier allyl addition. European Journal of Organic Chemistry 1999, (7), 1551-1560.

46. Vogel, A. I. Vogel's Elementary Practical Organic Chemistry. 3rd Ed. 1979; p 350 pp.

47. Turhanen, P. A.; Ahlgren, M. J.; Jarvinen, T.; Vepsalainen, J. J. Bisphosphonate prodrugs. Synthesis and identification of (1-hydroxyethylidene)-1,1-bisphosphonic acid tetraesters by mass spectrometry, NMR spectroscopy and X-ray crystallography. Phosphorus, Sulfur and Silicon and the Related Elements 2001, 170, 115-133.

All publications cited herein are incorporated by reference in their entirety.

What is claimed is:

1. A method of preparing a compound, comprising contacting iPr₄COBP with a Grignard reagent to form an alkyl or aryl substituted α-HRMBP ester, wherein the Grignard reagent is generated in Et₂O:THF, and wherein the ratio of Et₂O:THF is in the range of 5:1 to 1:5.

2. The method of claim 1, further comprising converting the alkyl or aryl substituted α-HRMBP ester into its corresponding acid or salt.

3. The method of claim 1, wherein the ratio of Et₂O:THF is 1:1.

4. The method of claim 1, wherein the Grignard reagent is RMgX, wherein R is an alkyl or aryl group and X is a halo group.

5. The method of claim 4, wherein R is a methyl, phenyl, or benzyl group.

6. A method of preparing a compound, comprising contacting iPr₄COBP or COTEPA with a Grignard reagent followed by an anion-interceptor to form a substituted α-HRMBP or α-HRMPA ester.

7. The method of claim 6, further comprising converting the substituted α-HRMBP or α-HRMPA ester into its corresponding acid or salt.

8. The method of claim 6, wherein the Grignard reagent is a heterocyclic Grignard reagent.

9. The method of claim 8, wherein the heterocyclic group contains nitrogen.

10. The method of claim 9, wherein the heterocyclic Grignard reagent is 2-chloropyridine Grignard reagent.

11. The method of claim 8, further comprising converting the substituted α-HRMBP or α-HRMPA ester into its corresponding acid in the presence of acetonitrile.

12. The method of claim 6, wherein the Grignard reagent is an alkyl or aryl Grignard reagent.

13. The method of claim 6, wherein the anion-interceptor is a silylating agent.

14. The method of claim 13, wherein the silylating agent is CTMS.

15. A substituted α-HRMBP or α-HRMPA ester prepared according to the method of claim 10, or the corresponding acid or salt.

16. A compound of formula

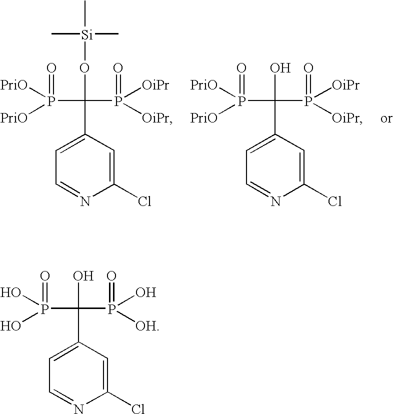

17. A method of preparing a compound, comprising contacting iPr₄COBP or COTEPA with an unsaturated indium halide reagent to form an unsaturated substituted α-HRMBP or α-HRMPA ester.

18. The method of claim 16, wherein the unsaturated indium halide reagent is allyl InBr₂.

19. The method of claim 18, wherein the allylic group is

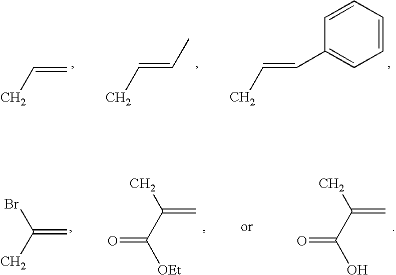

20. The method of claim 19, wherein 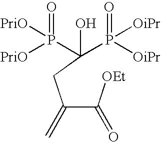
cyclizes to form

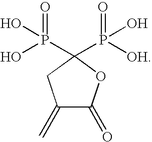

21. The method of claim 17, further comprising converting the unsaturated substituted α-HRMBP or α-HRMPA ester into its corresponding acid or salt.

22. The method of claim 21, wherein the unsaturated substituted α-HRMBP ester is converted into its corresponding acid in the presence of acetonitrile.

23. The method of claim 17, wherein iPr₄COBP or COTEPA is contacted with the unsaturated indium halide reagent in the presence of acetic acid or CTMS.

24. The method of claim 17, wherein iPr₄COBP or COTEPA is contacted with the unsaturated indium halide reagent in the presence of a Lewis acid.

25. The method of claim 24, wherein the Lewis acid is BF₃. etherate.

26. An unsaturated substituted α-HRMBP or α-HRMPA ester prepared according to the method of claim 17, or the corresponding acid or salt.

27. An unsaturated substituted α-HRMBP or α-HRMPA ester, or the corresponding acid or salt, wherein the unsaturated substitution group is

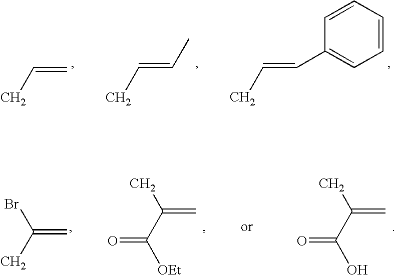

28. A compound of formula [chemical structure]
